SEQUENCE LISTING (76 sequences)

SEQ ID NO: 1 - protein, 8 aa, ARTIFICIAL SEQUENCE
  SEQUENCE FROM PHAGE LIBRARY
SYYPDFDY

SEQ ID NO: 2 - protein, 9 aa, ARTIFICIAL SEQUENCE
  SEQUENCE FROM PHAGE LIBRARY
DFLGYEFDY

SEQ ID NO: 3 - protein, 20 aa, ARTIFICIAL SEQUENCE
  SEQUENCE FROM PHAGE LIBRARY
YHSWYEMGYYGSTVGYMFDY

SEQ ID NO: 4 - protein, 10 aa, ARTIFICIAL SEQUENCE
  SEQUENCE FROM PHAGE LIBRARY
DNWFKPFSDV

SEQ ID NO: 5 - protein, 10 aa, ARTIFICIAL SEQUENCE
  SEQUENCE FROM PHAGE LIBRARY
VNHWTYTFDY

SEQ ID NO: 6 - protein, 17 aa, ARTIFICIAL SEQUENCE
  SEQUENCE FROM PHAGE LIBRARY
GYWYAYFTYINYGYFDN

SEQ ID NO: 7 - protein, 18 aa, ARTIFICIAL SEQUENCE
  SEQUENCE FROM PHAGE LIBRARY
TWQYSYFYYLDGGYYFDI

SEQ ID NO: 8 - protein, 17 aa, ARTIFICIAL SEQUENCE
  SEQUENCE FROM PHAGE LIBRARY
NMAYTNYQYVNMPHFDY

SEQ ID NO: 9 - protein, 17 aa, ARTIFICIAL SEQUENCE
  SEQUENCE FROM PHAGE LIBRARY
SMNSTMYWYLRRVLFDH

SEQ ID NO: 10 - protein, 9 aa, ARTIFICIAL SEQUENCE
  SEQUENCE FROM PHAGE LIBRARY
QSYDGPDLW

SEQ ID NO: 11 - protein, 9 aa, ARTIFICIAL SEQUENCE
  SEQUENCE FROM PHAGE LIBRARY
QSYDYSADY

SEQ ID NO: 12 - protein, 8 aa, ARTIFICIAL SEQUENCE
  SEQUENCE FROM PHAGE LIBRARY
QSYDFDFA

SEQ ID NO: 13 - protein, 8 aa, ARTIFICIAL SEQUENCE
  SEQUENCE FROM PHAGE LIBRARY
QQYDSIPY

SEQ ID NO: 14 - protein, 8 aa, ARTIFICIAL SEQUENCE
  SEQUENCE FROM PHAGE LIBRARY
QQMSNYPD

SEQ ID NO: 15 - protein, 9 aa, ARTIFICIAL SEQUENCE
  SEQUENCE FROM PHAGE LIBRARY
QSYDNNSDV

SEQ ID NO: 16 - protein, 8 aa, ARTIFICIAL SEQUENCE
  SEQUENCE FROM PHAGE LIBRARY
QQTNNAPV

SEQ ID NO: 17 - protein, 8 aa, ARTIFICIAL SEQUENCE
  SEQUENCE FROM PHAGE LIBRARY
QSYDYFKL

SEQ ID NO: 18 - protein, 9 aa, ARTIFICIAL SEQUENCE
  SEQUENCE FROM PHAGE LIBRARY
QSYDMYMYI

SEQ ID NO: 19 - protein, 120 aa, ARTIFICIAL SEQUENCE
  SEQUENCE FROM PHAGE LIBRARY
QVQLQQSGPGLVKPSQTLSLTCAISGDSVSSNSAAWNWIRQSPGRGLEWLGRTYYRSKWYNDYAVSVKSRITINPDTSKNQFSLQLNSVTPEDTAVYYCARSYYPDFDYWGQGTLVTVSS

SEQ ID NO: 20 - protein, 118 aa, ARTIFICIAL SEQUENCE
  SEQUENCE FROM PHAGE LIBRARY
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCARDFLGYEFDYWGQGTLVTVSS

SEQ ID NO: 21 - protein, 130 aa, ARTIFICIAL SEQUENCE
  SEQUENCE FROM PHAGE LIBRARY
QVQLKESGPALVKPTQTLTLTCTFSGFSLSTSGVGVGWIRQPPGKALEWLALIDWDDDKYYSTSLKTRLTISKDTSKNQVVLTMTNMDPVDTATYYCARYHSWYEMGYYGSTVGYMFDYWGQGTLVTVSS

SEQ ID NO: 22 - protein, 118 aa, ARTIFICIAL SEQUENCE
  SEQUENCE FROM PHAGE LIBRARY
QVQLVQSGAEVKKPGSSVKVSCKASGGTFSSYAISWVRQAPGQGLEWMGGIIPIFGTANYAQKFQGRTITADESTSTAYMELSSLRSEDTAVYYCARDNWFKPFSDVWGQGTLVTVSS

SEQ ID NO: 23 - protein, 119 aa, ARTIFICIAL SEQUENCE
  SEQUENCE FROM PHAGE LIBRARY
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARVNHWTYTFDYWGQGTLVTVSS

SEQ ID NO: 24 - protein, 126 aa, ARTIFICIAL SEQUENCE
  SEQUENCE FROM PHAGE LIBRARY
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCARGYWYAYFTYINYGYFDNWGQGTLVTVSS

SEQ ID NO: 25 - protein, 127 aa, ARTIFICIAL SEQUENCE
  SEQUENCE FROM PHAGE LIBRARY
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCARTWQYSYFYYLDGGYYFDIWGQGTLVTVSS

SEQ ID NO: 26 - protein, 126 aa, ARTIFICIAL SEQUENCE
  SEQUENCE FROM PHAGE LIBRARY
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCARNMAYTNYQYVNMPHFDYWGQGTLVTVSS

SEQ ID NO: 27 - protein, 126 aa, ARTIFICIAL SEQUENCE
  SEQUENCE FROM PHAGE LIBRARY
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCARSMNSTMYWYLRRVLFDHWGQGTLVTVSS

SEQ ID NO: 28 - protein, 109 aa, ARTIFICIAL SEQUENCE
  SEQUENCE FROM PHAGE LIBRARY
DIELTQPPSVSVAPGQTARISCSGDALGDKYASWYQQKPGQAPVLVIYDDSDRPSGIPERFSGSNSGNTATLTISGTQAEDEADYYCQSYDGPDLWVFGGGTKLTVLGQ

SEQ ID NO: 29 - protein, 109 aa, ARTIFICIAL SEQUENCE
  SEQUENCE FROM PHAGE LIBRARY
DIELTQPPSVSVAPGQTARISCSGDALGDKYASWYQQKPGQAPVLVIYDDSDRPSGIPERFSGSNSGNTATLTISGTQAEDEADYYCQSYDYSADYVFGGGTKLTVLGQ

SEQ ID NO: 30 - protein, 107 aa, ARTIFICIAL SEQUENCE
  SEQUENCE FROM PHAGE LIBRARY
DIELTQPPSVSVAPGQTARISCSGDALGDKYASWYQQKPGQAPVLVIYDDSDRPSGIPERFSGSNSGNTATLTISGTQAEDEADYYCQSYDFDFAVFGGTKLTVLGQ

SEQ ID NO: 31 - protein, 115 aa, ARTIFICIAL SEQUENCE
  SEQUENCE FROM PHAGE LIBRARY
DIVMTQSPDSLAVSLGERATINCRSSQSVLYSSNNKNYLAWYQQKPGQPPKLLIYWASTRESGVPDRFSGSGSGTDFTLTISSLQAEDVAVYYCQQYDSIPYTFGQGTKVEIKRT

SEQ ID NO: 32 - protein, 110 aa, ARTIFICIAL SEQUENCE
  SEQUENCE FROM PHAGE LIBRARY
DIVLTQSPATLSLSPGERATLSCRASQSVSSSYLAWYQQKPGQAPRLLIYGASSRATGVPARFSGSGSGTDFTLTISSLEPEDFATYYCQQMSNYPDTFGQGTKVEIKRT

SEQ ID NO: 33 - protein, 112 aa, ARTIFICIAL SEQUENCE
  SEQUENCE FROM PHAGE LIBRARY
DIALTQPASVSGSPGQSITISCTGTSSDVGGYNYVSWYQQHPGKAPKLMIYDVSNRPSGVSNRFSGSKSGNTASLTISGLQAEDEADYYCQSYDNNSDVVFGGGTKLTVLGQ

SEQ ID NO: 34 - protein, 110 aa, ARTIFICIAL SEQUENCE
  SEQUENCE FROM PHAGE LIBRARY
DIVLTQSPATLSLSPGERATLSCRASQSVSSSYLAWYQQKPGQAPRLLIYGASSRATGVPARFSGSGSGTDFTLTISSLEPEDFATYYCQQTNNAPVTFGQGTKVEIKRT

SEQ ID NO: 35 - protein, 108 aa, ARTIFICIAL SEQUENCE
  SEQUENCE FROM PHAGE LIBRARY
DIELTQPPSVSVAPGQTARISCSGDALGDKYASWYQQKPGQAPVLVIYDDSDRPSGIPERFSGSNSGNTATLTISGTQAEDEADYYCQSYDYFKLVFGGGTKLTVLGQ

SEQ ID NO: 36 - protein, 112 aa, ARTIFICIAL SEQUENCE
  SEQUENCE FROM PHAGE LIBRARY
DIALTQPASVSGSPGQSITISCTGTSSDVGGYNYVSWYQQHPGKAPKLMIYDVSNRPSGVSNRFSGSKSGNTASLTISGLQAEDEADYYCQSYDMYNYIVFGGGTKLTVLGQ

SEQ ID NO: 37 - protein, 249 aa, ARTIFICIAL SEQUENCE
  SEQUENCE FROM PHAGE LIBRARY
MLTCAISGNSVSSNSAAWNWIRQSPGRGLEWLGRTYYRSKWYNDYAVSVKSRITINPDTSKNQFSLQLNSVTPEDTAVYYCARSYYPDFDYWGQGTLVTVSSAGGGSGGGGSGGGGSGGGGSDIELTQPPSVSVAPGQTARISCSGDALGDKYASWYQQKPGQAPVLVIYDDSDRPSGIPERFSGSNSGNTATLTISGTQAEDEADYYCQSYDGPDLWVFGGGTKLTVLGQEFDYKMTMTKRAVEPPAV

SEQ ID NO: 38 - DNA, 750 bp, ARTIFICIAL SEQUENCE
  SEQUENCE FROM PHAGE LIBRARY
atgctgacctgtgcgatttccgggaatagcgtgagcagcaacagcgcggcgtggaactggattcgccagtctcctgggcgtggcctcgagtggctgggccgtacctattatcgtagcaaatggtataacgattatgcggtgagcgtgaaaagccggattaccatcaacccggatacttcgaaaaaccagtttagcctgcaactgaacagcgtgaccccggaagatacggccgtgtattattgcgcgcgttcttattatcctgattttgattattggggccaaggcaccctggtgacggttagctcagcgggtggcggttctggcggcggtgggagcggtggcggtggttctggcggtggtggttccgatatcgaactgacccagccgccttcagtgagcgttgcaccaggtcagaccgcgcgtatctcgtgtagcggcgatgcgctgggcgataaatacgcgagctggtaccagcagaaacccgggcaggcgccagttctggtgatttatgatgattctgaccgtccctcaggcatcccggaacgctttagcggatccaacagcggcaacaccgcgaccctgaccattagcggcactcaggcggaagacgaagcggattattattgccagagctatgacggtcctgatctttgggtgtttggcggcggcacgaagttaaccgttcttggccaggaattcgactataagatgacgatgacaaagcgcgccgtggagccacccgcagtttga

SEQ ID NO: 39 - DNA, 24 bp, ARTIFICIAL SEQUENCE
  SEQUENCE FROM PHAGE LIBRARY
tcttattatcctgattttgattat

SEQ ID NO: 40 - DNA, 27 bp, ARTIFICIAL SEQUENCE
  SEQUENCE FROM PHAGE LIBRARY
gattttcttggttatgagtttgattat

SEQ ID NO: 41 - DNA, 90 bp, ARTIFICIAL SEQUENCE
  SEQUENCE FROM PHAGE LIBRARY
tatcattcttggtatgagatgggttattatggttctactgttggttatatgtttgattatgataattggtttaagcctttttctgatgtt

SEQ ID NO: 42 - DNA, 30 bp, ARTIFICIAL SEQUENCE
  SEQUENCE FROM PHAGE LIBRARY
gataattggtttaagcctttttctgatgtt

SEQ ID NO: 43 - DNA, 30 bp, ARTIFICIAL SEQUENCE
  SEQUENCE FROM PHAGE LIBRARY
gttaatcattggacttatacttttgattat

SEQ ID NO: 44 - DNA, 51 bp, ARTIFICIAL SEQUENCE
  SEQUENCE FROM PHAGE LIBRARY
ggttattggtatgcttattttacttatattaattatggttattttgataat

SEQ ID NO: 45 - DNA, 54 bp, ARTIFICIAL SEQUENCE
  SEQUENCE FROM PHAGE LIBRARY
acttggcagtattcttatttttattatcttgatggtggttattattttgatatt

SEQ ID NO: 46 - DNA, 51 bp, ARTIFICIAL SEQUENCE
  SEQUENCE FROM PHAGE LIBRARY
aatatggcttatactaattatcagtatgttaatatgcctcattttgattat

SEQ ID NO: 47 - DNA, 51 bp, ARTIFICIAL SEQUENCE
  SEQUENCE FROM PHAGE LIBRARY
tctatgaattctactatgtattggtatcttcgtcgtgttctttttgatcat

SEQ ID NO: 48 - DNA, 27 bp, ARTIFICIAL SEQUENCE
  SEQUENCE FROM PHAGE LIBRARY
cagagctatgacggtcctgatctttgg

SEQ ID NO: 49 - DNA, 27 bp, ARTIFICIAL SEQUENCE
  SEQUENCE FROM PHAGE LIBRARY
cagagctatgactattctgctgattat

SEQ ID NO: 50 - DNA, 24 bp, ARTIFICIAL SEQUENCE
  SEQUENCE FROM PHAGE LIBRARY
cagagctatgactttgattttgct

SEQ ID NO: 51 - DNA, 24 bp, ARTIFICIAL SEQUENCE
  SEQUENCE FROM PHAGE LIBRARY
cagcagtatgattctattccttat

SEQ ID NO: 52 - DNA, 24 bp, ARTIFICIAL SEQUENCE
  SEQUENCE FROM PHAGE LIBRARY
cagcagatgtctaattatcctgat

SEQ ID NO: 53 - DNA, 27 bp, ARTIFICIAL SEQUENCE
  SEQUENCE FROM PHAGE LIBRARY
cagagctatgacaataattctgatgtt

SEQ ID NO: 54 - DNA, 24 bp, ARTIFICIAL SEQUENCE
  SEQUENCE FROM PHAGE LIBRARY
cagcagactaataatgctcctgtt

SEQ ID NO: 55 - DNA, 24 bp, ARTIFICIAL SEQUENCE
  SEQUENCE FROM PHAGE LIBRARY
cagagctatgactattttaagctt

SEQ ID NO: 56 - DNA, 27 bp, ARTIFICIAL SEQUENCE
  SEQUENCE FROM PHAGE LIBRARY
cagagctatgacatgtataattatatt

SEQ ID NO: 57 - DNA, 362 bp, ARTIFICIAL SEQUENCE
  SEQUENCE FROM PHAGE LIBRARY
caggtgcaattgcaacagtctggtccgggcctggtgaaaccgagccaaaccctgagcctgacctgtgcgatttccggagatagcgtgagcagcaacagcgcggcgtggaactggattcgccagtctcctgggcgtggcctcgagtggctgggccgtacctattatcgtagcaaatggtataacgattatgcggtgagcgtgaaaagccggattaccatcaacccggatacttcgaaaaaccagtttagcctgcaactgaacagcgtgaccccggaagatacggccgtgtattattgcgcgcgttcttattatcctgattttgattattggggccaaggcaccctggtgacggttagctcagc

SEQ ID NO: 58 - DNA, 356 bp, ARTIFICIAL SEQUENCE
  SEQUENCE FROM PHAGE LIBRARY
caggtgcaattggttcagagcggcgcggaagtgaaaaaaccgggcgcgagcgtgaaagtgagctgcaaagcctccggatatacctttaccagctattatatgcactgggtccgccaagcccctgggcagggtctcgagtggatgggctggattaacccgaatagcggcggcacgaactacgcgcagaagtttcagggccgggtgaccatgacccgtgataccagcattagcaccgcgtatatggaactgagcagcctgcgtagcgaagatacggccgtgtattattgcgcgcgtgattttcttggttatgagtttgattattggggccaaggcaccctggtgacggttagctcagc

SEQ ID NO: 59 - DNA, 392 bp, ARTIFICIAL SEQUENCE
  SEQUENCE FROM PHAGE LIBRARY
caggtgcaattgaaagaaagcggcccggccctggtgaaaccgacccaaaccctgaccctgacctgtaccttttccggatttagcctgtccacgtctggcgttggcgtgggctggattcgccagccgcctgggaaagccctcgagtggctggctctgattgattgggatgatgataagtattatagcaccagcctgaaaacgcgtctgaccattagcaaagatacttcgaaaaatcaggtggtgctgactatgaccaacatggacccggtggatacggccacctattattgcgcgcgttatcattcttggtatgagatgggttattatggttctactgttggttatatgtttgattattggggccaaggcaccctggtgacggttagctcagc

SEQ ID NO: 60 - DNA, 359 bp, ARTIFICIAL SEQUENCE
  SEQUENCE FROM PHAGE LIBRARY
caggtgcaattggttcagtctggcgcggaagtgaaaaaaccgggcagcagcgtgaaagtgagctgcaaagcctccggaggcacttttagcagctatgcgattagctgggtgcgccaagcccctgggcagggtctcgagtggatgggcggcattattccgatttttggcacggcgaactacgcgcagaagtttcagggccgggtgaccattaccgcggatgaaagcaccagcaccgcgtatatggaactgagcagcctgcgtagcgaagatacggccgtgtattattgcgcgcgtgataattggtttaagcctttttctgatgtttggggccaaggcaccctggtgacggttagctcagc

SEQ ID NO: 61 - DNA, 359 bp, ARTIFICIAL SEQUENCE
  SEQUENCE FROM PHAGE LIBRARY
caggtgcaattggttcagtctggcgcggaagtgaaaaaaccgggcagcagcgtgaaagtgagctgcaaagcctccggaggcacttttagcagctatgcgattagctgggtgcgccaagcccctgggcagggtctcgagtggatgggcggcattattccgatttttggcacggcgaactacgcgcagaagtttcagggccgggtgaccattaccgcggatgaaagcaccagcaccgcgtatatggaactgagcagcctgcgtagcgaagatacggccgtgtattattgcgcgcgtgttaatcattggacttatacttttgattattggggccaaggcaccctggtgacggttagctcagc

SEQ ID NO: 62 - DNA, 380 bp, ARTIFICIAL SEQUENCE
  SEQUENCE FROM PHAGE LIBRARY
caggtgcaattggttcagagcggcgcggaagtgaaaaaaccgggcgcgagcgtgaaagtgagctgcaaagcctccggatatacctttaccagctattatatgcactgggtccgccaagcccctgggcagggtctcgagtggatgggctggattaacccgaatagcggcggcacgaactacgcgcagaagtttcagggccgggtgaccatgacccgtgataccagcattagcaccgcgtatatggaactgagcagcctgcgtagcgaagatacggccgtgtattattgcgcgcgtggttattggtatgcttattttacttatattaattatggttattttgataattggggccaaggcaccctggtgacggttagctcagc

SEQ ID NO: 63 - DNA, 383 bp, ARTIFICIAL SEQUENCE
  SEQUENCE FROM PHAGE LIBRARY
caggtgcaattggttcagagcggcgcggaagtgaaaaaaccgggcgcgagcgtgaaagtgagctgcaaagcctccggatatacctttaccagctattatatgcactgggtccgccaagcccctgggcagggtctcgagtggatgggctggattaacccgaatagcggcggcacgaactacgcgcagaagtttcagggccgggtgaccatgacccgtgataccagcattagcaccgcgtatatggaactgagcagcctgcgtagcgaagatacggccgtgtattattgcgcgcgtacttggcagtattcttatttttattatcttgatggtggttattattttgatatttggggccaaggcaccctggtgacggttagctcagc

SEQ ID NO: 64 - DNA, 380 bp, ARTIFICIAL SEQUENCE
  SEQUENCE FROM PHAGE LIBRARY
caggtgcaattggttcagagcggcgcggaagtgaaaaaaccgggcgcgagcgtgaaagtgagctgcaaagcctccggatatacctttaccagctattatatgcactgggtccgccaagcccctgggcagggtctcgagtggatgggctggattaacccgaatagcggcggcacgaactacgcgcagaagtttcagggccgggtgaccatgacccgtgataccagcattagcaccgcgtatatggaactgagcagcctgcgtagcgaagatacggccgtgtattattgcgcgcgtaatatggcttatactaattatcagtatgttaatatgcctcattttgattattggggccaaggcaccctggtgacggttagctcagc

SEQ ID NO: 65 - DNA, 380 bp, ARTIFICIAL SEQUENCE
  SEQUENCE FROM PHAGE LIBRARY
caggtgcaattggttcagagcggcgcggaagtgaaaaaaccgggcgcgagcgtgaaagtgagctgcaaagcctccggatatacctttaccagctattatatgcactgggtccgccaagcccctgggcagggtctcgagtggatgggctggattaacccgaatagcggcggcacgaactacgcgcagaagtttcagggccgggtgaccatgacccgtgataccagcattagcaccgcgtatatggaactgagcagcctgcgtagcgaagatacggccgtgtattattgcgcgcgttctatgaattctactatgtattggtatcttcgtcgtgttctttttgatcattggggccaaggcaccctggtgacggttagctcagc

SEQ ID NO: 66 - DNA, 327 bp, ARTIFICIAL SEQUENCE
  SEQUENCE FROM PHAGE LIBRARY
gatatcgaactgacccagccgccttcagtgagcgttgcaccaggtcagaccgcgcgtatctcgtgtagcggcgatgcgctgggcgataaatacgcgagctggtaccagcagaaacccgggcaggcgccagttctggtgatttatgatgattctgaccgtccctcaggcatcccggaacgctttagcggatccaacagcggcaacaccgcgaccctgaccattagcggcactcaggcggaagacgaagcggattattattgccagagctatgacggtcctgatctttgggtgtttggcggcggcacgaagttaaccgttcttggccag

SEQ ID NO: 67 - DNA, 327 bp, ARTIFICIAL SEQUENCE
  SEQUENCE FROM PHAGE LIBRARY
gatatcgaactgacccagccgccttcagtgagcgttgcaccaggtcagaccgcgcgtatctcgtgtagcggcgatgcgctgggcgataaatacgcgagctggtaccagcagaaacccgggcaggcgccagttctggtgatttatgatgattctgaccgtccctcaggcatcccggaacgctttagcggatccaacagcggcaacaccgcgaccctgaccattagcggcactcaggcggaagacgaagcggattattattgccagagctatgactattctgctgattatgtgtttggcggcggcacgaagttaaccgttcttggccag

SEQ ID NO: 68 - DNA, 324 bp, ARTIFICIAL SEQUENCE
  SEQUENCE FROM PHAGE LIBRARY
gatatcgaactgacccagccgccttcagtgagcgttgcaccaggtcagaccgcgcgtatctcgtgtagcggcgatgcgctgggcgataaatacgcgagctggtaccagcagaaacccgggcaggcgccagttctggtgatttatgatgattctgaccgtccctcaggcatcccggaacgctttagcggatccaacagcggcaacaccgcgaccctgaccattagcggcactcaggcggaagacgaagcggattattattgccagagctatgactttgattttgctgtgtttggcggcggcacgaagttaaccgttcttggccag

SEQ ID NO: 69 - DNA, 345 bp, ARTIFICIAL SEQUENCE
  SEQUENCE FROM PHAGE LIBRARY
gatatcgtgatgacccagagcccggatagcctggcggtgagcctgggcgaacgtgcgaccattaactgcagaagcagccagagcgtgctgtatagcagcaacaacaaaaactatctggcgtggtaccagcagaaaccaggtcagccgccgaaactattaatttattgggcatccacccgtgaaagcggggtcccggatcgttttagcggctctggatccggcactgattttaccctgaccatttcgtccctgcaagctgaagacgtggcggtgtattattgccagcagtatgattctattccttatacctttggccagggtacgaaagttgaaattaaacgtacg

SEQ ID NO: 70 - DNA, 330 bp, ARTIFICIAL SEQUENCE
  SEQUENCE FROM PHAGE LIBRARY
gatatcgtgctgacccagagcccggcgaccctgagcctgtctccgggcgaacgtgcgaccctgagctgcagagcgagccagagcgtgagcagcagctatctggcgtggtaccagcagaaaccaggtcaagcaccgcgtctattaatttatggcgcgagcagccgtgcaactggggtcccggcgcgttttagcggctctggatccggcacggattttaccctgaccattagcagcctggaacctgaagactttgcgacttattattgccagcagatgtctaattatcctgatacctttggccagggtacgaaagttgaaattaaacgtacg

SEQ ID NO: 71 - DNA, 336 bp, ARTIFICIAL SEQUENCE
  SEQUENCE FROM PHAGE LIBRARY
gatatcgcactgacccagccagcttcagtgagcggctcaccaggtcagagcattaccatctcgtgtacgggtactagcagcgatgtgggcggctataactatgtgagctggtaccagcagcatcccgggaaggcgccgaaactgatgatttatgatgtgagcaaccgtccctcaggcgtgagcaaccgttttagcggatccaaaagcggcaacaccgcgagcctgaccattagcggcctgcaagcggaagacgaagcggattattattgccagagctatgacaataattctgatgttgtgtttggcggcggcacgaagttaaccgttcttggccag

SEQ ID NO: 72 - DNA, 330 bp, ARTIFICIAL SEQUENCE
  SEQUENCE FROM PHAGE LIBRARY
gatatcgtgctgacccagagcccggcgaccctgagcctgtctccgggcgaacgtgcgaccctgagctgcagagcgagccagagcgtgagcagcagctatctggcgtggtaccagcagaaaccaggtcaagcaccgcgtctattaatttatggcgcgagcagccgtgcaactggggtcccggcgcgttttagcggctctggatccggcacggattttaccctgaccattagcagcctggaacctgaagactttgcgacttattattgccagcagactaataatgctcctgttacctttggccagggtacgaaagttgaaattaaacgtacg

SEQ ID NO: 73 - DNA, 324 bp, ARTIFICIAL SEQUENCE
  SEQUENCE FROM PHAGE LIBRARY
gatatcgaactgacccagccgccttcagtgagcgttgcaccaggtcagaccgcgcgtatctcgtgtagcggcgatgcgctgggcgataaatacgcgagctggtaccagcagaaacccgggcaggcgccagttctggtgatttatgatgattctgaccgtccctcaggcatcccggaacgctttagcggatccaacagcggcaacaccgcgaccctgaccattagcggcactcaggcggaagacgaagcggattattattgccagagctatgactattttaagcttgtgtttggcggcggcacgaagttaaccgttcttggccag

SEQ ID NO: 74 - DNA, 336 bp, ARTIFICIAL SEQUENCE
  SEQUENCE FROM PHAGE LIBRARY
gatatcgcactgacccagccagcttcagtgagcggctcaccaggtcagagcattaccatctcgtgtacgggtactagcagcgatgtgggcggctataactatgtgagctggtaccagcagcatcccgggaaggcgccgaaactgatgatttatgatgtgagcaaccgtccctcaggcgtgagcaaccgttttagcggatccaaaagcggcaacaccgcgagcctgaccattagcggcctgcaagcggaagacgaagcggattattattgccagagctatgacatgtataattatattgtgtttggcggcggcacgaagttaaccgttcttggccag

SEQ ID NO: 75 - protein, 694 aa, HOMO SAPIENS
  ref <308>: DATABASE ACCESSION NUMBER: NCBI/P22607
  ref <309>: DATABASE ENTRY DATE: 1991-08-01
  ref <313>: RELEVANT RESIDUES: (1)..(375)
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 76 - DNA, 2085 bp, HOMO SAPIENS
  ref <308>: DATABASE ACCESSION NUMBER: NCBI/AF2451114
  ref <309>: DATABASE ENTRY DATE: 2002-03-28
  ref <313>: RELEVANT RESIDUES: (40)..(2124)
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaacggcagcaaggtgggcccggacggcacaccctacgttaccgtgctcaaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacaaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgactccgtgtttgcccacgacctgctgcccccggccccacccagcagtgggggctcgcggacgtga

(19) United States
(12) Patent Application Publication
(10) Pub. No.: US 2008/0044419 A1
(43) Pub. Date: Feb. 21, 2008

(54) Treatment of T Cell Mediated Diseases by Inhibition of Fgfr3

(75) Inventor: Avner Yayon, Moshav Sitria (IL)

Correspondence Address: BROWDY AND NEIMARK, P.L.L.C.;624 NINTH STREET, NW, SUITE 300, WASHINGTON, DC, 20001-5303, US

(73) Assignee: Fibron Ltd., Rehovot (IL)

(21) Appl. No.: 10/561,396

(22) PCT Filed: Jun. 17, 2004
(86) PCT No.: PCT/IL04/00528

§ 371 Date: Mar. 12, 2007

Related U.S. Application Data
(60) Provisional application No. 60/490,961, filed on Jul. 30, 2003.

(30) Foreign Application Priority Data
Jun. 17, 2003 (IL) 156495

(51) Int. Cl. A61K 39/395; A61K 38/00; A61P 1/00; A61P 19/02; A61P 3/10; A61P 17/06; A61K 39/00

(52) U.S. Cl. 424/139.1; 424/130.1; 424/143.1; 424/184.1; 514/2; 514/789

(57) ABSTRACT

The present invention provides a method of preventing and treating a T cell mediated disease, including inflammatory autoimmune diseases and in particular rheumatoid arthritis, by administering to an individual in need thereof at least one FGFR 3 inhibitor including a molecule comprising the antigen-binding portion of an antibody having a specific affinity for fibroblast growth factor receptor 3 (FGFR3), a FGFR3 specific small organic molecule tyrosine kinase inhibitor, a FGFR3 specific soluble receptor, a FGFR3 peptide or peptidomimetic, a FGFR3 specific RNA inhibitor, a FGFR3 specific antagonist ligand or a DNA vaccine encoding FGFR3 or a fragment thereof, or an inhibitor of heparan sulfate binding.

### FIELD OF THE INVENTION

The present invention relates to a method of preventing and treating T cell mediated diseases, including inflammatory autoimmune diseases and in particular rheumatoid arthritis, by administering to an individual in need thereof at least one inhibitor of fibroblast growth factor receptor 3 (FGFR3).

### BACKGROUND OF THE INVENTION

### T Cell Mediated Disease

The human immune system is a highly regulated cellular network that normally functions to defend the body from infection. In some instances, the immune system malfunctions and reacts to a host component as if it were foreign. Such a response results in an autoimmune disease, in which the host's immune system mistakenly attacks self, targeting the host's own tissue. T cells, the primary regulators of the immune system, directly or indirectly effect the autoimmune response. T cell-mediated diseases refer to any disease directly mediated by T cells and those indirectly mediated whereby a T cell response contributes to the production of abnormal antibodies and frequently an inflammatory response.

Aberrant immune mechanisms are believed to result in more than eighty diseases, among these are rheumatoid arthritis (RA), systemic lupus erythematosus (SLE), multiple sclerosis (MS), type I diabetes (juvenile onset, insulin mediated diabetes mellitus, IDDM), myasthenia gravis (MG) and psoriasis. Autoimmune diseases affect millions of people worldwide. In addition to the individual suffering generated by these diseases, the cost in terms of actual treatment expenditures and lost productivity is measured in billions of dollars annually.

### Rheumatoid Arthritis

Rheumatoid arthritis (RA) is a systemic inflammatory autoimmune disease that primarily affects the small diarthrodial joints of the hands and feet. The disease manifests itself as inflammation of the normally acellular synovium, the joint lining, due to the infiltration of macrophages, T cells and other immune cells into the region. T cells appear to play an important role in the etiopathogenesis of RA since T-cell cytokines such as interleukin-2 (IL-2) and interferon-γ are present in the synovium and synovial fluid. Damage to the joints depends on three primary elements: inflammation, infiltration of the synovial tissue with immune cells and angiogenesis. The locally expressed degradative enzymes and cytokines digest the extracellular matrix and destroy the articular tissue resulting in chronic pain and irreversible damage of tendons, ligaments, joints, and bones. RA affects approximately 0.5-1% of the adult population in the western world.

### Biological Therapy for T Cell Mediated Disease

Novel biological approaches for treating autoimmune disease, including monoclonal antibodies, soluble receptors, and enzyme inhibitors, target pro-inflammatory cytokines, their cell surface receptors and cell types, have emerged as a result of the recent advances in understanding the physiopathology underlying inflammation. (reviewed in Smolen and Steiner, 2003; Nepom, 2002; Simón, 2001).

U.S. Pat. No. 5,919,452 teaches the treatment of tumor necrosis factor a (TNFα) mediated pathologies such as autoimmune and inflammatory autoimmune diseases by administering compounds such as anti-TNFα antibodies and anti-TNFα peptides. U.S. Pat. No. 5,512,544 discloses the treatment of autoimmune disease comprising administering TNFα binding proteins, which are in effect soluble forms of the TNF receptor, to an individual in need thereof. U.S. Pat. No. 6,333,032 discloses a method of treating an autoimmune disease in a human patient comprising administering an antibody to γ-interferon.

U.S. Pat. No. 6,730,666, by some of the inventors of the present invention, teaches a method of inhibiting growth factor receptor tyrosine kinase activity in an individual in need thereof by administering a composition comprising a porphyrin or a derivative thereof. According to one embodiment of that invention, the compositions may be useful for the treatment rheumatoid arthritis and other autoimmune diseases, specifically by inhibiting VEGF (vascular endothelial growth factor) and NGF (nerve growth factor).

PCT application WO 01/57056 discloses a method for treating RA in an individual, comprising the step of expressing within the individual at least an immunologically recognizable portion of a cytokine from an exogenous polynucleotide wherein a level of expression of the at least a portion of the cytokine is sufficient to induce the formation of anti-cytokine immunoglobulins which serve to neutralize or ameliorate the activity of a respective endogenous cytokine. Expression of a molecule for inhibiting FGFR was neither taught nor suggested.

The use of anti-angiogenic compounds, specifically those which inhibit VEGF signaling, for the treatment of RA has been proposed (reviewed in Paleolog, 2002) based on the rationale that disruption of new blood vessel formation would prevent delivery of nutrients to the inflammatory site and may lead to vessel regression.

### Fibroblast Growth Factors

Fibroblast Growth Factors (FGFs) constitute a family of over twenty structurally related polypeptides that are developmentally regulated and expressed in a wide variety of tissues. FGFs stimulate proliferation, cell migration and differentiation and play a major role in skeletal and limb development, wound healing, tissue repair, hematopoiesis, angiogenesis, and tumorigenesis (reviewed in Ornitz and Itoh, 2001).

The biological action of FGFs is mediated by specific cell surface receptors belonging to the receptor protein tyrosine kinase (RPTK) family of protein kinases. These proteins consist of an extracellular ligand binding domain, a single transmembrane domain and an intracellular tyrosine kinase domain that undergoes phosphorylation upon binding of FGF. The FGF receptor (FGFR) extracellular region contains three immunoglobulin-like (Ig-like) loops or domains (D1, D2 and D3), an acidic box, and a heparin-binding domain. Four FGFR genes encoding for multiple receptor variants have been identified to date.

A role for the FGF system and receptors in T cell mediated disease has yet to be defined. An increase in endogenous production of FGF2 in the synovial fluid of RA patients may contribute to joint destruction by inducing osteoclastogenesis (Manabe, et al., 1999). A neutralizing anti-FGF2 antibody was shown to inhibit osteoclastogenesis induced by RA synovial fluid in a cell culture system (Manabe, et al., 1999) and to attenuate the clinical symptoms and histopathological abnormalities in a rat adjuvant induced arthritis (AIA) model (Yamashita, et al., 2002). Japanese patent publication 2002-229883 discloses a bFGF (FGF2) antagonist as a therapeutic agent for treating chronic rheumatoid arthritis.

PCT publication WO 02/102973, co-assigned to the assignee of the present invention, discloses antibodies to receptor tyrosine kinases, specifically anti-Fibroblast Growth Factor Receptor 3 (FGFR3) antibodies. Certain antibodies shown to be specific for FGFR3 are useful to neutralize FGFR3 activity and for the treatment of skeletal dysplasias such as achondroplasia and proliferative diseases such as bladder cancer and multiple myeloma.

International patent application WO 03/023004 discloses antisense modulation of GFR3 expression for the treatment of skeletal and proliferative disorders. T cell mediated diseases are neither taught nor suggested.

The art neither teaches nor suggests the use of FGFR3 inhibitors, including anti-FGFR3 antibodies, for the prevention or treatment of T cell mediated autoimmune inflammatory diseases.

Citation of any document herein is not intended as an admission that such document is pertinent prior art, or considered material to the patentability of any claim of the present application.

### SUMMARY OF THE INVENTION

It is now disclosed that the inhibition of fibroblast growth factor receptor 3 (FGFR3) activity represents for the first time a novel and unexpected means of treating T cell mediated inflammatory or autoimmune diseases. The present invention relates to the prevention, attenuation or treatment of an autoimmune disease by administering a therapeutically effective amount of at least one FGFR3 inhibitor to a mammal in need thereof. The methods of the present invention are effective in treatment of T-cell mediated inflammatory or autoimmune diseases including but not limited to rheumatoid arthritis (RA), collagen II arthritis, multiple sclerosis (MS), systemic lupus erythematosus (SLE), psoriasis, juvenile onset diabetes, Sjogren's disease, thyroid disease, sarcoidosis, autoimmune uveitis, inflammatory bowel disease (Crohn's and ulcerative colitis), celiac disease and myasthenia gravis.

In one aspect the present invention is related to a method of preventing, attenuating or treating the symptoms of a T-cell mediated inflammatory or autoimmune disease comprising administering a pharmaceutical composition comprising a therapeutically effective amount of at least one FGFR3 inhibitor and a pharmaceutically acceptable carrier. Another aspect relates to the use of at least one FGFR3 inhibitor for the manufacture of a medicament for the treatment of a T cell mediated inflammatory autoimmune disease.

An inhibitor may be selected from diverse types of compounds. According to certain embodiments a FGFR3 inhibitor is a molecule comprising at least the antigen-binding portion of an antibody having a specific affinity for fibroblast growth factor receptor 3 (FGFR3). According to other embodiments a FGFR3 specific inhibitor useful in treating a T cell mediated inflammatory or autoimmune disease is selected from a FGFR3 specific small organic molecule tyrosine kinase inhibitor, a FGFR3 specific soluble receptor, a FGFR3 peptide or peptidomimetic, a FGFR3 specific RNA inhibitor, a FGFR3 specific antagonist ligand or a DNA vaccine encoding FGFR3 or a fragment thereof, an FGFR3 specific inhibitor of heparan sulfate binding.

In one embodiment, the present invention provides a method of treating a T cell mediated inflammatory autoimmune disease by administering a pharmaceutical composition comprising at least one therapeutically effective FGFR3 inhibitor selected from the group consisting of a molecule comprising at least the antigen-binding portion of an antibody having a specific affinity for fibroblast growth factor receptor 3 (FGFR3), a FGFR3 specific small organic molecule tyrosine kinase inhibitor, a FGFR3 specific soluble receptor, a FGFR3 specific peptide or peptidomimetic, a FGFR3 specific RNA inhibitor, a FGFR3 specific antagonist ligand and a DNA vaccine encoding FGFR3 or a fragment thereof, an FGFR3 specific inhibitor of heparin binding.

In another embodiment, the present invention provides the use of a FGFR3 inhibitor selected from the group consisting of a molecule comprising the antigen-binding portion of an antibody which has a specific affinity for fibroblast growth factor receptor 3 (FGFR3), a FGFR3 specific small organic molecule tyrosine kinase inhibitor, a FGFR3 specific soluble receptor, a FGFR3 specific peptide or peptidomimetic, a FGFR3 specific RNA inhibitor, a FGFR3 specific antagonist ligand and a DNA vaccine encoding FGFR3 or a fragment thereof, an FGFR3 specific inhibitor of heparin binding, for the manufacture of a medicament for the prevention and treatment of a T cell mediated inflammatory autoimmune disease.

According to certain embodiments of the present invention the FGFR3 inhibitor is a molecule that comprises the antigen-binding portion of an antibody which has a specific affinity for fibroblast growth factor receptor 3 (FGFR3). In a preferred embodiment the molecule comprises the antigen-binding portion of an antibody which has specific affinity for the extracellular domain of fibroblast growth factor receptor 3 (FGFR3).

According to certain embodiments the antibody is polyclonal, monoclonal, or proteolytic fragments thereof such as the Fab or F(ab′)₂ fragments. Additional embodiments include chimeric antibodies; human and humanized antibodies; recombinant and engineered antibodies, and fragments thereof. A preferred antibody species is a single chain antibody. Single chain antibodies can be single chain composite polypeptides having antigen binding capabilities and comprising amino acid sequences homologous or analogous to the variable regions of an immunoglobulin light and heavy chain i.e. linked V_{H}-V_{L} or single chain Fv (scFv).

Some of the molecules and compositions thereof described herein have been disclosed in PCT patent application WO 02/102972, the teachings of which are incorporated by reference as if fully set forth herein, co-assigned to the assignee of the present invention. These compositions were disclosed previously as being useful for treating skeletal dysplasias and proliferative diseases.

It is now disclosed that said known compositions are unexpectedly useful for the treatment of a T cell mediated inflammatory or autoimmune disease including but not limited to rheumatoid arthritis, collagen II arthritis, multiple sclerosis, systemic lupus erythematosus, psoriasis, juvenile onset diabetes, Sjogren's disease, thyroid disease, sarcoidosis, celiac disease, autoimmune uveitis, inflammatory bowel disease (Crohn's and ulcerative colitis) and myasthenia gravis.

In certain embodiments the present invention provides a method of treating or preventing a T cell mediated inflammatory or autoimmune disease comprising administering a composition comprising a therapeutically effective molecule comprising a V_{H}-CDR3 region having a polypeptide sequence as set forth in anyone of SEQ ID NOS: 1-9 and a V_{L}-CDR3 region having a polypeptide sequence as set forth in anyone of SEQ ID NOS: 10-18, and a pharmaceutically acceptable carrier. The corresponding polynucleotide sequences of the V_{H}-CDR3 and V_{L}-CDR3 regions are set forth in SEQ ID NOS: 39-47 and SEQ ID NOS: 48-56, respectively. These sequences were disclosed previously in WO 02/102972, assigned to some of the applicants of the present invention.

According to various additional embodiments the present invention provides a method of treating or preventing a T cell mediated inflammatory or autoimmune disease comprising administering a composition comprising a therapeutically effective molecule comprising a V_{H} domain having a polypeptide sequence as set forth in anyone of SEQ ID NOS: 19-27 and the V_{L} domains having a polypeptide sequence as set forth in anyone of SEQ ID NOS: 28-36, and a pharmaceutically acceptable carrier. The corresponding polynucleotide sequences of the V_{H} and V_{L} domains are set forth in SEQ ID NOS: 57-65 and SEQ ID NOS: 66-74, respectively.

In a preferred embodiment, the present invention provides a method of treating or preventing a T cell mediated inflammatory or autoimmune disease comprising administering a pharmaceutical composition which comprises a molecule comprising a V_{H}-CDR3 region and a V_{L}-CDR3 region set forth in SEQ ID NO:1 and SEQ ID NO:10, respectively, and a pharmaceutically acceptable carrier. In another preferred embodiment the pharmaceutical composition comprises a V_{H} domain and a V_{L} domain set forth in SEQ ID NO: 19 and SEQ ID NO:27, respectively, and a pharmaceutically acceptable carrier. In yet another preferred embodiment the pharmaceutical composition comprises a single chain Fv molecule (scFv) set forth in SEQ ID NO:37, having corresponding polynucleotide sequence SEQ ID NO:38, and a pharmaceutically acceptable carrier.

In another embodiment, the present invention provides a method of treating a T cell mediated inflammatory or autoimmune disease by administering a composition comprising at least one FGFR3 inhibitor wherein the inhibitor is a FGFR3 specific small organic molecule tyrosine kinase inhibitor, and a pharmaceutically acceptable carrier. According to certain embodiments of the present invention a small molecule tyrosine kinase (TK) inhibitor having FGFR3 specificity is useful for preventing, attenuating or treating a T cell mediated inflammatory autoimmune disease.

In yet another embodiment, the present invention provides a method of treating a T cell mediated inflammatory autoimmune disease by administering a pharmaceutical composition comprising at least one FGFR3 inhibitor wherein the inhibitor is a FGFR3 specific soluble receptor, and a pharmaceutically acceptable carrier. In a non-limiting example, a secreted receptor, also known as a soluble receptor, comprises the extracellular ligand-binding portion of the FGFR3 receptor, per se or fused to the constant region (Fc) of a human immunoglobulin (Ig) chain.

In yet another embodiment, the present invention provides a method of treating a T cell mediated inflammatory or autoimmune disease by administering a pharmaceutical composition comprising at least one FGFR3 inhibitor wherein the inhibitor is a FGFR3 specific peptide inhibitor, and a pharmaceutically acceptable carrier. A peptide inhibitor includes FGFR3 specific peptides, peptide analogs having amino acid sequence derived from the extracellular portion of the fibroblast growth factor receptor 3 (FGFR3) and peptidomimetics based on the structure of such peptides.

In yet another embodiment, the present invention provides a method of treating a T cell mediated inflammatory or autoimmune disease by administering a pharmaceutical composition comprising at least one FGFR3 inhibitor wherein the inhibitor is a FGFR3 specific antagonist ligand, and a pharmaceutically acceptable carrier. Non-limiting examples of ligands antagonistic to FGFR3 may be found in copending PCT publication WO 03/094835, assigned to the assignee of the present invention.

In yet another embodiment, the present invention provides a method of treating a T cell mediated inflammatory or autoimmune disease by administering a pharmaceutical composition comprising at least one FGFR3 inhibitor wherein the inhibitor is a FGFR3 specific RNA inhibitor, and a pharmaceutically acceptable carrier. RNA inhibition (RNAi) is based on antisense modulation of FGFR3 in cells and tissues comprising contacting the cells and tissues with at least one antisense molecule, including but not limited to double stranded RNA, (dsRNA), small interfering RNA (siRNA), ribozymes and locked nucleic acids (LNAs), and a pharmaceutically acceptable carrier. In certain specific embodiments the RNA inhibiting molecule is an antisense oligonucleotide or an oligonucleotide mimetic comprising from about 8 to about 50 nucleotides.

In another embodiment, the present invention provides a method of treating a T cell mediated inflammatory or autoimmune disease by administering a pharmaceutical composition comprising at least one FGFR3 inhibitor wherein the inhibitor is a DNA vaccine encoding FGFR3 or a fragment thereof, and a pharmaceutically acceptable carrier. DNA vaccination provides an effective means of long term antigen expression in vivo for the generation of both humoral and cellular immune responses. According to various embodiments of the present invention the DNA vaccines encode active fragments of FGFR3. The preferred fragments are polynucleotides encoding the extracellular domain of FGFR3, in particular amino acids about 1-370 or fragments thereof. The FGFR3 according to the present invention is preferably human, however other mammalian FGFR3 proteins are within the scope of the invention.

### BRIEF DESCRIPTION OF THE FIGURES

FIG. 1 shows the effect of certain FGFR3 inhibitors on limb swelling in a murine Collagen Induced Arthritis (CIA) model.

FIG. 2 depicts the effect of certain FGFR3 inhibitors in a delayed type hypersensitivity assay in a murine model.

FIG. 3 shows the interferon gamma secretion of lymphocytes isolated from FGFR3 inhibitor treated and non-treated animals.

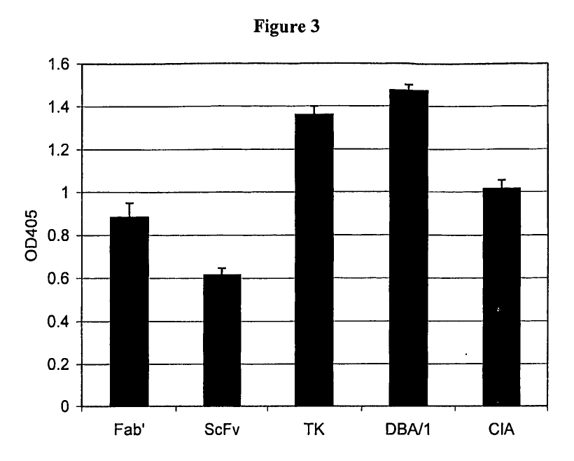

### DETAILED DESCRIPTION OF THE INVENTION

The present invention is based on the discovery that inhibition or attenuation of fibroblast growth factor receptor 3 (FGFR3)-mediated signaling results in the prevention and treatment of T cell mediated inflammatory or autoimmune diseases including, but not limited to, rheumatoid arthritis. It is now disclosed that the inhibition of fibroblast growth factor receptor 3 (FGFR3) represents a novel and unexpected means of treating a T cell mediated inflammatory or autoimmune disease. The present invention relates to the prevention, attenuation or treatment of autoimmune disease by administering a therapeutically effective amount of at least one FGFR3 inhibitor to a mammal in need thereof. The methods of the present invention are effective in treatment of T-cell mediated inflammatory or autoimmune disease including, but not limited to, rheumatoid arthritis, collagen II arthritis, multiple sclerosis, systemic lupus erythematosus, psoriasis, juvenile onset diabetes, Sjogren's disease, thyroid disease, sarcoidosis, celiac disease, autoimmune uveitis, inflammatory bowel disease (Crohn's and ulcerative colitis) and myasthenia gravis.

The present invention further relates to the use of at least one FGFR3 inhibitor for the manufacture of a medicament for the prevention, attenuation or treatment of a T cell mediated inflammatory or autoimmune disease, including but not limited to rheumatoid arthritis.

FGFR3 inhibitors may be selected from diverse types of compounds and molecules and may inhibit FGFR3 signaling by a variety of means. Without wishing to be bound to any particular theory, a FGFR3 inhibitor may inhibit FGFR3 activity by transcriptional, post-transcriptional, translational or post translational mechanisms known to inhibit receptor tyrosine kinase activity. The inhibitor may inhibit FGFR3 activity by interfering at the extracellular or intracellular level and may inhibit any known FGFR3 isoform.

In a non-limiting example, a FGFR3 inhibitor may be an antigen binding molecule of different types such as polyclonal or monoclonal antibodies or a monoclonal antibody fragment. It may take the form of a FGFR3 soluble receptor per se or the form of a soluble receptor fusion protein, for example a fusion protein using a Fc fragment. A FGFR3 inhibitor may be a FGFR3 specific small organic molecule tyrosine kinase inhibitor, a FGFR3 specific peptide or peptidomimetic, a FGFR3 specific RNA inhibitor, FGFR3 specific ligand antagonist and a DNA vaccine encoding FGFR3 or a fragment thereof.

PCT publication WO 02/102972, co-assigned to the assignee of the present invention, discloses monoclonal antibodies to receptor protein tyrosine kinases, including specific anti-Fibroblast Growth Factor Receptor 3 (FGFR3) antibodies. Certain antibodies were shown to be specific for FGFR3 and useful to neutralize FGFR3 activity and for the treatment of skeletal dysplasias such as achondroplasia and proliferative diseases such as bladder cancer and multiple myeloma. Utilizing a soluble dimeric form of the extracellular domain of the FGFR3 receptor to screen for antibodies (e.g., Fabs) from a phage display antibody library yielded numerous high affinity (K_{D}<50 nM) antibodies (Fabs) that bind FGFR3 and interfere with ligand binding, thereby blocking ligand-dependent activation of FGFR3. Additional antibodies useful for blocking ligand-independent, or constitutive, activation were also identified and isolated.

The present inventors have now discovered that certain molecules disclosed in that application are useful for the prevention and treatment of T cell mediated inflammatory or autoimmune diseases, including but not limited to rheumatoid arthritis, collagen II arthritis, multiple sclerosis, systemic lupus erythematosus, psoriasis, juvenile onset diabetes, Sjogren's disease, thyroid disease, sarcoidosis, autoimmune uveitis, inflammatory bowel disease (Crohn's and ulcerative colitis), celiac disease and myasthenia gravis.

For convenience certain terms employed in the specification, examples and claims are described herein.

The term “fibroblast growth factor receptor” or “FGFR” denotes a receptor specific for FGF which is necessary for transducing the signal exerted by FGF to the cell interior, typically comprising an extracellular ligand-binding domain, a single transmembrane helix, and a cytoplasmic domain having tyrosine kinase activity. The FGFR extracellular domain consists of three immunoglobulin-like (Ig-like) domains (D1, D2 and D3), a heparin binding domain and an acidic box. Four FGFR genes that encode for multiple receptor protein variants are known. Alternative splicing of the FGFR3 mRNAs generates at least two known isoforms of the receptors, FGFR3IIIc and FGFR3IIIb.

Throughout the specification and the claims that follow, the term “FGFR3 specific” refers to any effector that has higher affinity or activity or binding to FGFR3 polypeptide or to the polynucleotide encoding same, than to another FGF receptor protein or polynucleotide. The effector can be any molecule including a ligand, an inhibitor, an antibody, a polypeptide, a polynucleotide or a small organic molecule such as a tyrosine kinase inhibitor. It is to be explicitly understood that the term “FGFR3 specific” does not exclude or preclude situations wherein the effector has some activity on another FGF receptor subtype. It is further to be understood that if the activity mediated via another receptor subtype is clinically important for the therapeutic utility observed, this is explicitly encompassed within the scope of the claimed invention.

As used herein “T cell mediated inflammatory or autoimmune diseases” are diseases directly or indirectly effected by the T cells of the immune system. Non-limitative examples include rheumatoid arthritis, collagen II arthritis, multiple sclerosis, systemic lupus erythematosus, psoriasis, juvenile onset diabetes, Sjogren's disease, thyroid disease, sarcoidosis, autoimmune uveitis, inflammatory bowel disease (Crohn's and ulcerative colitis), celiac disease and myasthenia gravis.

One aspect of the present invention is directed to a method of preventing, attenuating or treating a T cell mediated inflammatory or autoimmune disease by administering a molecule comprising the antigen-binding portion of an antibody which diminishes or inhibits activation of FGFR3, and a pharmaceutically acceptable carrier. According to one embodiment of the present invention the antigen-binding portion of an antibody is directed to the extracellular domain of the FGFR3.

One embodiment of the present invention is directed to molecules comprising an antigen binding domain which blocks ligand-dependent activation of FGFR3.

The molecule having the antigen-binding portion of an antibody according to the present invention is useful for blocking the ligand-dependent activation and/or ligand independent (constitutive) activation of FGFR3. Preferred embodiments of such antibodies/molecules, obtained from an antibody library designated as HuCAL® (Human Combinatorial Antibody Library) clone, are presented in Table 1 with the unique V_{H}-CDR3 and V_{L}-CDR3 sequences presented in Table 2.

TABLE 1
Properties of antibodies useful for inhibiting, treating or attenuating
T cell mediated inflammatory or autoimmune diseases.

| | Affinity to | Affinity to | | | | |
|---|---|---|---|---|---|---|
| | FGFR3 | FGFR3 | Affinity to | | IC₅₀ FGFR3 | |
| | (BIAcore) | (FACS) | FGFR1 | K_{off} | (FGF9) | Domain |
| Clone | nM | nM | nM | (s⁻¹) | nM | Specificity |
| PRO-001 | 1.5 | 0.7 | — | 7.1 × 10e−4 | 19 | 2 |
| PRO-002 | 37 | 43 | — | 2 × 10e−2 | 360 | 2 |
| PRO-012 | 14 | 6.5 | — | 2.3 × 10e−3 | 58 | 2 |
| PRO-021 | 9 | 1.1 | — | 3.6 × 10e−3 | 50 | 3c |
| PRO-024 | 10 | NA | — | 5.4 × 10e−3 | 70 | 3c |
| PRO-026 | 4 | 1.4 | 32 | 5 × 10e−4 | 70 | 3c |
| PRO-029 | 6 | <1 | 29 | 1.4 × 10e−3 | 20 | 3c |
| PRO-054 | 3.7 | NA | 2.5 | 2 × 10e−3 | 45 | 3c |
| PRO-055 | 2.9 | NA | — | 7.4 × 10e−4 | 34 | 3c |

Key: affinity (nM) of the respective molecules to FGFR3 and FGFR1 was measured by BIAcore and/or FACS. IC₅₀ were determined for the dimeric dHLX format of certain molecule with antigen binding site in an FDCP-FGFR3 proliferation assay performed with FGF9. Fab-dHLX refers to a Fab mini-antibody format where a dimer of the Fab monomer is produced as a fusion protein after insertion into an expression vector.

TABLE 2

V_{H}-CDR3 and corresponding V_{L}-CDR3 polypeptide
sequences

| | | |
|---|---|---|
| Clone | V_{H}-CDR3 | V_{L}-CDR3 |

| | | |
|---|---|---|
| PRO-001 | SYYPDFDY | QSYDGPDLW |
| | (SEQ ID NO:1) | (SEQ ID NO:10) |
| PRO-002 | DFLGYEFDY | QSYDYSADY |
| | (SEQ ID NO:2) | (SEQ ID NO:11) |
| PRO-012 | YHSWYEMGYY GSTVGYMFDY | QSYDFDFA |
| | (SEQ ID NO:3) | (SEQ ID NO:12) |
| PRO-021 | DNWFKPFSDV | QQYDSIPY |
| | (SEQ ID NO:4) | (SEQ ID NO:13) |
| PRO-024 | VNHWTYTFDY | QQMSNYPD |
| | (SEQ ID NO:5) | (SEQ ID NO:14) |
| PRO-026 | GYWYAYFTYI NYGYFDN | QSYDNNSDV |
| | (SEQ ID NO:6) | (SEQ ID NO:15) |
| PRO-029 | TWQYSYFYYL DGGYYFDI | QQTNNAPV |
| | (SEQ ID NO:7) | (SEQ ID NO:16) |
| PRO-054 | NMAYTNYQYV NMPHFDY | QSYDYFKL |
| | (SEQ ID NO:8) | (SEQ ID NO:17) |
| PRO-055 | SMNSTMYWYL RRVLFDH | QSYDMYMYI |
| | (SEQ ID NO:9) | (SEQ ID NO:18) |

V_{H} refers to the variable heavy chain, V_{L} refers to the variable light chain, CDR3 refers to complementarity determining region 3. In certain preferred embodiments the present invention provides a method of treating or preventing T cell mediated inflammatory or autoimmune disease comprising administering a composition a composition comprising a therapeutically effective molecule comprising a V_{H}-CDR3 region having a polypeptide sequence as set forth in any one of SEQ ID NOS: 1-9 and a corresponding V_{L}-CDR3 region having a polypeptide sequence as set forth in any one of SEQ ID NOS:10-18, and a pharmaceutically acceptable carrier. The corresponding polynucleotide sequences of the V_{H}-CDR3 and V_{L}-CDR3 regions as set forth in any one of SEQ ID NOS: 39-47 and SEQ ID NOS: 48-56, respectively. The polynucleotide sequences are presented in Table 3.

According to certain embodiments the present invention provides a method of treating or preventing T cell mediated inflammatory or autoimmune disease comprising administering a composition comprising a therapeutically effective molecule comprising a V_{H} domain having a polypeptide sequence as set forth in any one of SEQ ID NOS: 19-27 and the corresponding V_{L} domains having a polypeptide sequence as set forth in any one of SEQ ID NOS: 28-36, and a pharmaceutically acceptable carrier. The preferred V_{H} and V_{L} sequences are presented herein.

| PRO-001-VH (SEQ ID NO:19) | |
|---|---|
| 1 | QVQLQQSGPG LVKPSQTLSL TCAISGDSVS SNSAAWNWIR QSPGRGLEWL |
| 51 | GRTYYRSKWY NDYAVSVKSR ITINPDTSKN QFSLQLNSVT PEDTAVYYCA |
| 101 | RSYYPDFDYW GQGTLVTVSS |
| PRO-002-VH (SEQ ID NO:20) | |
| 1 | QVQLVQSGAE VKKPGASVKV SCKASGYTFT SYYMHWVRQA PGQGLEWMGW |
| 51 | INPNSGGTNY AQKFQGRVTM TRDTSISTAY MELSSLRSED TAVYYCARDF |
| 101 | LGYEFDYWGQ GTLVTVSS |
| PRO-012-VH (SEQ ID NO:21) | |
| 1 | QVQLKESGPA LVKPTQTLTL TCTFSGFSLS TSGVGVGWIR QPPGKALEWL |
| 51 | ALIDWDDDKY YSTSLKTRLT ISKDTSKNQV VLTMTNMDPV DTATYYCARY |
| 101 | HSWYEMGYYG STVGYMFDYW GQGTLVTVSS |
| PRO-021-VH (SEQ ID NO:22) | |
| 1 | QVQLVQSGAE VKKPGSSVKV SCKASGGTFS SYAISWVRQA PGQGLEWMGG |
| 51 | IIPIFGTANY AQKFQGRVTI TADESTSTAY MELSSLRSED TAVYYCARDN |
| 101 | WFKPFSDVWG QGTLVTVSS |
| PRO-024-VH (SEQ ID NO:23) | |
| 1 | QVQLVQSGAE VKKPGSSVKV SCKASGGTFS SYAISWVRQA PGQGLEWMGG |
| 51 | IIPIFGTANY AQKFQGRVTI TADESTSTAY MELSSLRSED TAVYYCARVN |
| 101 | HWTYTFDYWG QGTLVTVSS |
| MS-Pro-26-VH (SEQ ID NO:24) | |
| 1 | QVQLVQSGAE VKKPGASVKV SCKASGYTFT SYYMHWVRQA PGQGLEWMGW |
| 51 | INPNSGGTNY AQKFQGRVTM TRDTSISTAY MELSSLRSED TAVYYCARGY |
| 101 | WYAYFTYINY GYFDNWGQGT LVTVSS |
| PRO-029-VH (SEQ ID NO:25) | |
| 1 | QVQLVQSGAE VKKPGASVKV SCKASGYTFT SYYMHWVRQA PGQGLEWMGW |
| 51 | INPNSGGTNY AQKFQGRVTM TRDTSISTAY MELSSLRSED TAVYYCARTW |
| 101 | QYSYFYYLDG GYYFDIWGQG TLVTVSS |
| PRO-054-VH (SEQ ID NO:26) | |
| 1 | QVQLVQSGAE VKKPGASVKV SCKASGYTFT SYYMHWVRQA PGQGLEWMGW |
| 51 | INPNSGGTNY AQKFQGRVTM TRDTSISTAY MELSSLRSED TAVYYCARNM |
| 101 | AYTNYQYVNM PHFDYWGQGT LVTVSS |
| PRO-055-VH (SEQ ID NO:27) | |
| 1 | QVQLVQSGAE VKKPGASVKV SCKASGYTFT SYYMHWVRQA PGQGLEWMGW |
| 51 | INPNSGGTNY AQKFQGRVTM TRDTSISTAY MELSSLRSED TAVYYCARSM |
| 101 | NSTMYWYLRR VLFDHWGQGT LVTVSS |
| PRO-001-VL (SEQ ID NO:28) | |
| 1 | DIELTQPPSV SVAPGQTARI SCSGDALGDK YASWYQQKPG QAPVLVIYDD |
| 51 | SDRPSGIPER FSGSNSGNTA TLTISGTQAE DEADYYCQSY DGPDLWVFGG |
| 101 | GTKLTVLGQ |
| PRO-002-VL (SEQ ID NO:29) | |
| 1 | DIELTQPPSV SVAPGQTARI SCSGDALGDK YASWYQQKPG QAPVLVIYDD |
| 51 | SDRPSGIPER FSGSNSGNTA TLTISGTQAE DEADYYCQSY DYSADYVFGG |
| 101 | GTKLTVLGQ |
| PRO-012-VL (SEQ ID NO:30) | |
| 1 | DIELTQPPSV SVAPGQTARI SCSGDALGDK YASWYQQKPG QAPVLVIYDD |
| 51 | SDRPSGIPER FSGSNSGNTA TLTISGTQAE DEADYYCQSY DFDFAVFGGG |
| 101 | TKLTVLGQ |
| PRO-021-VL (SEQ ID NO:31) | |
| 1 | DIVMTQSPDS LAVSLGERAT INCRSSQSVL YSSNNKNYLA WYQQKPGQPP |
| 51 | KLLIYWASTR ESGVPDRFSG SGSGTDFTLT ISSLQAEDVA VYYCQQYDSI |
| 101 | PYTFGQGTKV EIKRT |
| PRO-024-VL (SEQ ID NO:32) | |
| 1 | DIVLTQSPAT LSLSPGERAT LSCRASQSVS SSYLAWYQQK PGQAPRLLIY |
| 51 | GASSRATGVP ARFSGSGSGT DFTLTISSLE PEDFATYYCQ QMSNYPDTFG |
| 101 | QGTKVEIKRT |
| MS-Pro-26-VL (SEQ ID NO:33) | |
| 1 | DIALTQPASV SGSPGQSITI SCTGTSSDVG GYNYVSWYQQ HPGKAPKLMI |
| 51 | YDVSNRPSGV SNRFSGSKSG NTASLTISGL QAEDEADYYC QSYDNNSDVV |
| 101 | FGGGTKLTVL GQ |
| PRO-029-VL (SEQ ID NO:34) | |
| 1 | DIVLTQSPAT LSLSPGERAT LSCRASQSVS SSYLAWYQQK PGQAPRLLIY |
| 51 | GASSRATGVP ARFSGSGSGT DFTLTISSLE PEDFATYYCQ QTNNAPVTFG |
| 101 | QGTKVEIKRT |
| PRO-054-VL (SEQ ID NO:35) | |
| 1 | DIELTQPPSV SVAPGQTARI SCSGDALGDK YASWYQQKPG QAPVLVIYDD |
| 51 | SDRPSGIPER FSGSNSGNTA TLTISGTQAE DEADYYCQSY DYFKLVFGGG |
| 101 | TKLTVLGQ |
| PRO-055-VL (SEQ ID NO:36) | |
| 1 | DIALTQPASV SGSPGQSITI SCTGTSSDVG GYNYVSWYQQ HPGKAPKLMI |
| 51 | YDVSNRPSGV SNRFSGSKSG NTASLTISGL QAEDEADYYC QSYDMYNYIV |
| 101 | FGGGTKLTVL GQ |

The corresponding polynucleotide sequences of the V_{H} and V_{L} domains have SEQ ID NOS: 57-65 and SEQ ID NOS: 66-74, respectively.

<SEQ ID NO:57; DNA> PRO-001 VH
CAGGTGCAATTGCAACAGTCTGGTCCGGGCCTGGTGAAACCGAGCCAAACCCTGAGCCTGACCTGTG
CGATTTCCGGAGATAGCGTGAGCAGCAACAGCGCGGCGTGGAACTGGATTCGCCAGTCTCCTGGGCG
TGGCCTCGAGTGGCTGGGCCGTACCTATTATCGTAGCAAATGGTATAACGATTATGCGGTGAGCGTG
AAAAGCCGGATTACCATCAACCCGGATACTTCGAAAAACCAGTTTAGCCTGCAACTGAACAGCGTGA
CCCCGGAAGATACGGCCGTGTATTATTGCGCGCGTTCTTATTATCCTGATTTTGATTATTGGGGCCA
AGGCACCCTGGTGACGGTTAGCTCAGC
<SEQ ID NO:58; DNA> PRO-002 VH
CAGGTGCAATTGGTTCAGAGCGGCGCGGAAGTGAAAAAACCGGGCGCGAGCGTGAAAGTGAGCTGCA
AAGCCTCCGGATATACCTTTACCAGCTATTATATGCACTGGGTCCGCCAAGCCCCTGGGCAGGGTCT
CGAGTGGATGGGCTGGATTAACCCGAATAGCGGCGGCACGAACTACGCGCAGAAGTTTCAGGGCCG
GGTGACCATGACCCGTGATACCAGCATTAGCACCGCGTATATGGAACTGAGCAGCCTGCGTAGCGAA
GATACGGCCGTGTATTATTGCGCGCGTGATTTTCTTGGTTATGAGTTTGATTATTGGGGCCAAGGCA
CCCTGGTGACGGTTAGCTCAGC
<SEQ ID NO:59; DNA> PRO-012 VH
CAGGTGCAATTGAAAGAAAGCGGCCCGGCCCTGGTGAAACCGACCCAAACCCTGACCCTGACCTGTA
CCTTTTCCGGATTTAGCCTGTCCACGTCTGGCGTTGGCGTGGGCTGGATTCGCCAGCCGCCTGGGAA
AGCCCTCGAGTGGCTGGCTCTGATTGATTGGGATGATGATAAGTATTATAGCACCAGCCTGAAAAC
GCGTCTGACCATTAGCAAAGATACTTCGAAAAATCAGGTGGTGCTGACTATGACCAACATGGACCCG
GTGGATACGGCCACCTATTATTGCGCGCGTTATCATTCTTGGTATGAGATGGGTTATTATGGTTCTA
CTGTTGGTTATATGTTTGATTATTGGGGCCAAGGCACCCTGGTGACGGTTAGCTCAGC
<SEQ ID NO:60; DNA> PRO-021 VH
CAGGTGCAATTGGTTCAGTCTGGCGCGGAAGTGAAAAAACCGGGCAGCAGCGTGAAAGTGAGCTGCA
AAGCCTCCGGAGGCACTTTTAGCAGCTATGCGATTAGCTGGGTGCGCCAAGCCCCTGGGCAGGGTCT
CGAGTGGATGGGCGGCATTATTCCGATTTTTGGCACGGCGAACTACGCGCAGAAGTTTCAGGGCCGG
GTGACCATTACCGCGGATGAAAGCACCAGCACCGCGTATATGGAACTGAGCAGCCTGCGTAGCGAAG
ATACGGCCGTGTATTATTGCGCGCGTGATAATTGGTTTAAGCCTTTTTCTGATGTTTGGGGCCAAGG
CACCCTGGTGACGGTTAGCTCAGC
<SEQ ID NO:61; DNA> PRO-024 VH
CGTGAAAGTGAGCTGCAAAGCCTCCGGAGGCACTTTTAGCAGCTATGCGATTAGCTGGGTGCGCCAA
GCCCCTGGGCAGGGTCTCGAGTGGATGGGCGGCATTATTCCGATTTTTGGCACGGCGAACTACGCGC
AGAAGTTTCAGGGCCGGGTGACCATTACCGCGGATGAAAGCACCAGCACCGCGTATATGGAACTGAG
CAGCCTGCGTAGCGAAGATACGGCCGTGTATTATTGCGCGCGTGTTAATCATTGGACTTATACTTTT
GATTATTGGGGCCAAGGCACCCTGGTGACGGTTAGCTCAGC
<SEQ ID NO:62; DNA> PRO-026 VH
CGTGAAAGTGAGCTGCAAAGCCTCCGGATATACCTTTACCAGCTATTATATGCACTGGGTCCGCCAA
GCCCCTGGGCAGGGTCTCGAGTGGATGGGCTGGATTAACCCGAATAGCGGCGGCACGAACTACGCGC
AGAAGTTTCAGGGCCGGGTGACCATGACCCGTGATACCAGCATTAGCACCGCGTATATGGAACTGAG
CAGCCTGCGTAGCGAAGATACGGCCGTGTATTATTGCGCGCGTGGTTATTGGTATGCTTATTTTACT
TATATTAATTATGGTTATTTTGATAATTGGGGCCAAGGCACCCTGGTGACGGTTAGCTCAGC
<SEQ ID NO:63; DNA> PRO-029 VH
CAGGTGCAATTGGTTCAGAGCGGCGCGGAAGTGAAAAAACCGGGCGCGAGCGTGAAAGTGAGCTGCA
AAGCCTCCGGATATACCTTTACCAGCTATTATATGCACTGGGTCCGCCAAGCCCCTGGGCAGGGTCT
CGAGTGGATGGGCTGGATTAACCCGAATAGCGGCGGCACGAACTACGCGCAGAAGTTTCAGGGCCGG
GTGACCATGACCCGTGATACCAGCATTAGCACCGCGTATATGGAACTGAGCAGCCTGCGTAGCGAAG
ATACGGCCGTGTATTATTGCGCGCGTACTTGGCAGTATTCTTATTTTTATTATCTTGATGGTGGTTA
TTATTTTGATATTTGGGGCCAAGGCACCCTGGTGACGGTTAGCTCAGC
<SEQ ID NO:64; DNA> PRO-054 VH
CAGGTGCAATTGGTTCAGAGCGGCGCGGAAGTGAAAAAACCGGGCGCGAGCGTGAAAGTGAGCTGCA
AAGCCTCCGGATATACCTTTACCAGCTATTATATGCACTGGGTCCGCCAAGCCCCTGGGCAGGGTCT
CGAGTGGATGGGCTGGATTAACCCGAATAGCGGCGGCACGAACTACGCGCAGAAGTTTCAGGGCCGG
GTGACCATGACCCGTGATACCAGCATTAGCACCGCGTATATGGAACTGAGCAGCCTGCGTAGCGAAG
ATACGGCCGTGTATTATTGCGCGCGTAATATGGCTTATACTAATTATCAGTATGTTAATATGCCTCA
TTTTGATTATTGGGGCCAAGGCACCCTGGTGACGGTTAGCTCAGC
<SEQ ID NO:65; DNA> PRO-055 VH
CAGGTGCAATTGGTTCAGAGCGGCGCGGAAGTGAAAAAACCGGGCGCGAGCGTGAAAGTGAGCTGCA
AAGCCTCCGGATATACCTTTACCAGCTATTATATGCACTGGGTCCGCCAAGCCCCTGGGCAGGGTCT
CGAGTGGATGGGCTGGATTAACCCGAATAGCGGCGGCACGAACTACGCGCAGAAGTTTCAGGGCCGG
GTGACCATGACCCGTGATACCAGCATTAGCACCGCGTATATGGAACTGAGCAGCCTGCGTAGCGAAG
ATACGGCCGTGTATTATTGCGCGCGTTCTATGAATTCTACTATGTATTGGTATCTTCGTCGTGTTCT
TTTTGATCATTGGGGCCAAGGCACCCTGGTGACGGTTAGCTCAGC
<SEQ ID NO:66> PRO-001 VL
GATATCGAACTGACCCAGCCGCCTTCAGTGAGCGTTGCACCAGGTCAGACCGCGCGTATCTCGTGTA
GCGGCGATGCGCTGGGCGATAAATACGCGAGCTGGTACCAGCAGAAACCCGGGCAGGCGCCAGTTCT
GGTGATTTATGATGATTCTGACCGTCCCTCAGGCATCCCGGAACGCTTTAGCGGATCCAACAGCGGC
AACACCGCGACCCTGACCATTAGCGGCACTCAGGCGGAAGACGAAGCGGATTATTATTGCCAGAGCT
ATGACGGTCCTGATCTTTGGGTGTTTGGCGGCGGCACGAAGTTAACCGTTCTTGGCCAG
<SEQ ID NO:67; DNA> PRO-002 VL
GATATCGAACTGACCCAGCCGCCTTCAGTGAGCGTTGCACCAGGTCAGACCGCGCGTATCTCGTGTA
GCGGCGATGCGCTGGGCGATAAATACGCGAGCTGGTACCAGCAGAAACCCGGGCAGGCGCCAGTTCT
GGTGATTTATGATGATTCTGACCGTCCCTCAGGCATCCCGGAACGCTTTAGCGGATCCAACAGCGGC
AACACCGCGACCCTGACCATTAGCGGCACTCAGGCGGAAGACGAAGCGGATTATTATTGCCAGAGCT
ATGACTATTCTGCTGATTATGTGTTTGGCGGCGGCACGAAGTTAACCGTTCTTGGCCAG
<SEQ ID NO:68; DNA> PRO-012 VL
GATATCGAACTGACCCAGCCGCCTTCAGTGAGCGTTGCACCAGGTCAGACCGCGCGTATCTCGTGTA
GCGGCGATGCGCTGGGCGATAAATACGCGAGCTGGTACCAGCAGAAACCCGGGCAGGCGCCAGTTCT
GGTGATTTATGATGATTCTGACCGTCCCTCAGGCATCCCGGAACGCTTTAGCGGATCCAACAGCGGC
AACACCGCGACCCTGACCATTAGCGGCACTCAGGCGGAAGACGAAGCGGATTATTATTGCCAGAGCT
ATGACTTTGATTTTGCTGTGTTTGGCGGCGGCACGAAGTTAACCGTTCTTGGCCAG
<SEQ ID NO:69; DNA> PRO-021 VL
GATATCGTGATGACCCAGAGCCCGGATAGCCTGGCGGTGAGCCTGGGCGAACGTGCGACCATTAACT
GCAGAAGCAGCCAGAGCGTGCTGTATAGCAGCAACAACAAAAACTATCTGGCGTGGTACCAGCAGAA
ACCAGGTCAGCCGCCGAAACTATTAATTTATTGGGCATCCACCCGTGAAAGCGGGGTCCCGGATCGT
TTTAGCGGCTCTGGATCCGGCACTGATTTTACCCTGACCATTTCGTCCCTGCAAGCTGAAGACGTGG
CGGTGTATTATTGCCAGCAGTATGATTCTATTCCTTATACCTTTGGCCAGGGTACGAAAGTTGAAAT
TAAACGTACG
<SEQ ID NO:70; DNA> PRO-024 VL
GATATCGTGCTGACCCAGAGCCCGGCGACCCTGAGCCTGTCTCCGGGCGAACGTGCGACCCTGAGCT
GCAGAGCGAGCCAGAGCGTGAGCAGCAGCTATCTGGCGTGGTACCAGCAGAAACCAGGTCAAGCACC
GCGTCTATTAATTTATGGCGCGAGCAGCCGTGCAACTGGGGTCCCGGCGCGTTTTAGCGGCTCTGGA
TCCGGCACGGATTTTACCCTGACCATTAGCAGCCTGGAACCTCAAGACTTTGCGACTTATTATTGCC
AGCAGATGTCTAATTATCCTGATACCTTTGGCCAGGGTACGAAAGTTGAAATTAAACGTACG
<SEQ ID N0:71; DNA> PRO-026 VL
GATATCGCACTGACCCAGCCAGCTTCAGTGAGCGGCTCACCAGGTCAGAGCATTACCATCTCGTGTA
CGGGTACTAGCAGCGATGTGGGCGGCTATAACTATGTGAGCTGGTACCAGCAGCATCCCGGGAAGGC
GCCGAAACTGATGATTTATGATGTGAGCAACCGTCCCTCAGGCGTGAGCAACCGTTTTAGCGGATCC
AAAAGCGGCAACACCGCGAGCCTGACCATTAGCGGCCTGCAAGCGGAAGACGAAGCGGATTATTATT
GCCAGAGCTATGACAATAATTCTGATGTTGTGTTTGGCGGCGGCACGAAGTTAACCGTTCTTGGCCA
G
<SEQ ID N0:72; DNA> PRO-029 VL
GATATCGTGCTGACCCAGAGCCCGGCGACCCTGAGCCTGTCTCCGGGCGAACGTGCGACCCTGAGCT
GCAGAGCGAGCCAGAGCGTGAGCAGCAGCTATCTGGCGTGGTACCAGCAGAAACCAGGTCAAGCACC
GCGTCTATTAATTTATGGCGCGAGCAGCCGTGCAACTGGGGTCCCGGCGCGTTTTAGCGGCTCTGGA
TCCGGCACGGATTTTACCCTGACCATTAGCAGCCTGGAACCTGAAGACTTTGCGACTTATTATTGCC
AGCAGACTAATAATGCTCCTGTTACCTTTGGCCAGGGTACGAAAGTTGAAATTAAACGTACG
<SEQ ID NO:73; DNA> PRO-054 VL
GATATCGAACTGACCCAGCCGCCTTCAGTGAGCGTTGCACCAGGTCAGACCGCGCGTATCTCGTGTA
GCGGCGATGCGCTGGGCGATAAATACGCGAGCTGGTACCAGCAGAAACCCGGGCAGGCGCCAGTTCT
GGTGATTTATGATGATTCTGACCGTCCCTCAGGCATCCCGGAACGCTTTAGCGGATCCAACAGCGGC
AACACCGCGACCCTGACCATTAGCGGCACTCAGGCGGAAGACGAAGCGGATTATTATTGCCAGAGCT
ATGACTATTTTAAGCTTGTGTTTGGCGGCGGCACGAAGTTAACCGTTCTTGGCCAG
<SEQ ID N0:74; DNA> PRO-055 VL
GATATCGCACTGACCCAGCCAGCTTCAGTGAGCGGCTCACCAGGTCAGAGCATTACCATCTCGTGTA
CGGGTACTAGCAGCGATGTGGGCGGCTATAACTATGTGAGCTGGTACCAGCAGCATCCCGGGAAGGC
GCCGAAACTGATGATTTATGATGTGAGCAACCGTCCCTCAGGCGTGAGCAACCGTTTTAGCGGATCC
AAAAGCGGCAACACCGCGAGCCTGACCATTAGCGGCCTGCAAGCGGAAGACGAAGCGGATTATTATT
GCCAGAGCTATGACATGTATAATTATATTGTGTTTGGCGGCGGCACGAAGTTAACCGTTCTTGGCCA
G

In yet another preferred embodiment the pharmaceutical composition comprises a single chain Fv molecule (scFv) set forth in SEQ ID NO:37, having corresponding polynucleotide sequence SEQ ID NO:38, and a pharmaceutically acceptable carrier. The respective polypeptide and polynucleotide sequences are presented herein:

PRO-001 scFv polypeptide (SEQ ID NO:37)
MLTCAISGNS VSSNSAAWNW IRQSPGRGLE WLGRTYYRSK
WYNDYAVSVK SRITINPDTS KNQFSLQLNS VTPEDTAVYY
CARSYYPDFD YWGQGTLVTV SSAGGGSGGG GSGGGGSGGG
GSDIELTQPP SVSVAPGQTA RISCSGDALG DKYASWYQQK
PGQAPVLVIY DDSDRPSGIP ERFSGSNSGN TATLTISGTQ
AEDEADYYCQ SYDGPDLWVF GGGTKLTVLG QEFDYKMTMT
KPAVEPPAV
PRO-001 scFv DNA (SEQ ID NO:38)
1 ATGCTGACCT GTGCGATTTC CGGGAATAGC
GTGAGCAGCA ACAGCGCGGC GTGGAACTGG ATTCGCCAGT
CTCCTGGGCG TGGCCTCGAG TGGCTGGGCC GTACCTATTA
TCGTAGCAAA TGGTATAACG ATTATGCGGT GAGCGTGAAA
AGCCGGATTA CCATCAACCC GGATACTTCG AAAAACCAGT
TTAGCCTGCA ACTGAACAGC GTGACCCCGG AAGATACGGC
CGTGTATTAT TGCGCGCGTT CTTATTATCC TGATTTTGAT
TATTGGGGCC AAGGCACCCT GGTGACGGTT AGCTCAGCGG
GTGGCGGTTC TGGCGGCGGT GGGAGCGGTG GCGGTGGTTC
TGGCGGTGGT GGTTCCGATA TCGAACTGAC CCAGCCGCCT
TCAGTGAGCG TTGCACCAGG TCAGACCGCG CGTATCTCGT
GTAGCGGCGA TGCGCTGGGC GATAAATACG CGAGCTGGTA
CCAGCAGAAA CCCGGGCAGG CGCCAGTTCT GGTGATTTAT
GATGATTCTG ACCGTCCCTC AGGCATCCCG GAACGCTTTA
GCGGATCCAA CAGCGGCAAC ACCGCGACCC TGACCATTAG
CGGCACTCAG GCGGAAGACG AAGCGGATTA TTATTGCCAG
AGCTATGACG GTCCTGATCT TTGGGTGTTT GGCGGCGGCA
CGAAGTTAAC CGTTCTTGGC CAGGAATTCG ACTATAAGAT
GACGATGACA AAGCGCGCCG TGGAGCCACC CGCAGTTTGA

TABLE 3

V_{H}-CDR3 and corresponding V_{L}-CDR3
polynucleotide sequence

| | | |
|---|---|---|
| Clone | V_{H}-CDR3 | V_{L}-CDR3 |

| | | |
|---|---|---|
| PRO-001 | TCTTATTATC CTGATTTTGA | CAGAGCTATG ACGGTCCTGA |
| | TTAT | TCTTTGG |
| | (SEQ ID NO:39) | (SEQ ID NO:48) |
| PRO-002 | GATTTTCTTG GTTATGAGTT | CAGAGCTATG ACTATTCTGC |
| | TGATTAT | TGATTAT |
| | (SEQ ID NO:40) | (SEQ ID NO:49) |
| PRO-012 | TATCATTCTT GGTATGAGAT | CAGAGCTATG ACTTTGATTT |
| | GGGTTATTAT GGTTCTACTG | TGCT |
| | TTGGTTATAT GTTTGATTAT | (SEQ ID NO:50) |
| | (SEQ ID NO:41) | |
| PRO-021 | GATAATTGGT TTAAGCCTTT | CAGCAGTATG ATTCTATTCC |
| | TTCTGATGTT | TTAT |
| | (SEQ ID NO:42) | (SEQ ID NO:51) |
| PRO-024 | GTTAATCATT GGACTTATAC | CAGCAGATGT CTAATTATCC |
| | TTTTGATTAT | TGAT |
| | (SEQ ID NO:43) | (SEQ ID NO:52) |
| PRO-026 | GGTTATTGGT ATGCTTATTT | CAGAGCTATG ACAATAATTC |
| | TACTTATATT AATTATGGTT | TGATGTT |
| | ATTTTCATAAT | (SEQ ID NO:53) |
| | (SEQ ID NO:44) | |
| PRO-029 | ACTTGGCAGT ATTCTTATTT | CAGCAGACTA ATAATGCTCC |
| | TTATTATCTT GATGGTGGTT | TGTT |
| | ATTATTTTGA TATT | (SEQ ID NO:54) |
| | (SEQ ID NO:45) | |
| PRO-054 | AATATGGCTT ATACTAATTA | CAGAGCTATG ACTATTTTAA |
| | TCAGTATGTT AATATGCCTC | GCTT |
| | ATTTTGATTA T | (SEQ ID NO:55) |
| | (SEQ ID NO:46) | |
| PRO-055 | TCTATGAATT CTACTATGTAT | CAGAGCTATG ACATGTATAA |
| | TGGTATCTTC GTCGTGTTCTT | TTATATT |
| | TTTGATCAT | (SEQ ID NO:56) |
| | (SEQ ID NO:47) | |

### Antibodies

Antibodies, or immunoglobulins, comprise two heavy chains linked together by disulfide bonds and two light chains, each light chain being linked to a respective heavy chain by disulfide bonds in a “Y” shaped configuration. Proteolytic digestion of an antibody yields Fv (Fragment variable and Fc (fragment crystalline) domains. The antigen binding domains, Fab, include regions where the polypeptide sequence varies. The term F(ab′)₂ represents two Fab′ arms linked together by disulfide bonds. The central axis of the antibody is termed the Fc fragment. Each heavy chain has at one end a variable domain (V_{H}) followed by a number of constant domains (C_{H}). Each light chain has a variable domain (V_{L}) at one end and a constant domain (C_{L}) at its other end, the light chain variable domain being aligned with the variable domain of the heavy chain and the light chain constant domain being aligned with the first constant domain of the heavy chain (CHI).

The variable domains of each pair of light and heavy chains form the antigen-binding site. The domains on the light and heavy chains have the same general structure and each domain comprises four framework regions, whose sequences are relatively conserved, joined by three hypervariable domains known as complementarity determining regions (CDR1-3). These domains contribute specificity and affinity of the antigen-binding site.

The isotype of the heavy chain (gamma, alpha, delta, epsilon or mu) determines immunoglobulin class (IgG, IgA, IgD, IgE or IgM, respectively). The light chain is either of two isotypes (kappa, κ or lambda, λ) found in all antibody classes.

It should be understood that when the terms “antibody” or “antibodies” are used, this is intended to include intact antibodies, such as polyclonal antibodies or monoclonal antibodies (mAbs), as well as proteolytic fragments thereof such as the Fab or F(ab′)₂ fragments. Further included within the scope of the invention are chimeric antibodies; human and humanized antibodies; recombinant and engineered antibodies, and fragments thereof. Furthermore, the DNA encoding the variable region of the antibody can be inserted into the DNA encoding other antibodies to produce chimeric antibodies (see, for example, U.S. Pat. No. 4,816,567). Single chain antibodies fall within the scope of the present invention. Single chain antibodies can be single chain composite polypeptides having antigen binding capabilities and comprising amino acid sequences homologous or analogous to the variable regions of an immunoglobulin light and heavy chain (linked V_{H}-V_{L} or single chain Fv (ScFv)). Both V_{H} and V_{L} may copy natural monoclonal antibody sequences or one or both of the chains may comprise a CDR-FR construct of the type described in U.S. Pat. No. 5,091,513, the entire contents of which are incorporated herein by reference. The separate polypeptides analogous to the variable regions of the light and heavy chains are held together by a polypeptide linker. Methods of production of such single chain antibodies, particularly where the DNA encoding the polypeptide structures of the V_{H} and V_{L} chains are known, may be accomplished in accordance with the methods described, for example, in U.S. Pat. Nos. 4,946,778, 5,091,513 and 5,096,815, the entire contents of each of which are incorporated herein by reference.

Additionally, CDR grafting may be performed to alter certain properties of the antibody molecule including affinity or specificity. A non-limiting example of CDR grafting is disclosed in U.S. Pat. No. 5,225,539.

A “molecule having the antigen-binding portion of an antibody” as used herein is intended to include not only intact immunoglobulin molecules of any isotype and generated by any animal cell line or microorganism, but also the antigen-binding reactive fraction thereof, including, but not limited to, the Fab fragment, the Fab′ fragment, the F(ab′)₂ fragment, the variable portion of the heavy and/or light chains thereof, Fab miniantibodies (see WO 93/15210, U.S. patent application Ser. No. 08/256,790, WO 96/13583, U.S. patent application Ser. No. 08/817,788, WO 96/37621, U.S. patent application Ser. No. 08/999,554, the entire contents of which are incorporated herein by reference), dimeric bispecific miniantibodies (see Muller, et al, 1998) and chimeric or single-chain antibodies incorporating such reactive fraction, as well as any other type of molecule or cell in which such antibody reactive fraction has been physically inserted, such as a chimeric T-cell receptor or a T-cell having such a receptor, or molecules developed to deliver therapeutic moieties by means of a portion of the molecule containing such a reactive fraction. Such molecules may be provided by any known technique, including, but not limited to, enzymatic cleavage, peptide synthesis or recombinant techniques.

The term “Fc” as used herein is meant as that portion of an immunoglobulin molecule (Fragment crystallizable) that mediates phagocytosis, triggers inflammation and targets Ig to particular tissues; the Fc portion is also important in complement activation.

In one embodiment of the invention, a chimera comprising a fusion of the extracellular domain of the RPTK and an immunoglobulin constant domain can be constructed useful for assaying for ligands for the receptor and for screening for antibodies and fragments thereof

The “extracellular domain” when used herein refers the polypeptide sequence of the FGFR3 disclosed herein which are normally positioned to the outside of the cell. The extracellular domain encompasses polypeptide sequences in which part of or all of the adjacent (C-terminal) hydrophobic transmembrane and intracellular sequences of the mature FGFR3 have been deleted. Thus, the extracellular domain-containing polypeptide can comprise the extracellular domain and a part of the transmembrane domain. Alternatively, in the preferred embodiment, the polypeptide comprises only the extracellular domain of the FGFR3. The truncated extracellular domain is generally soluble. The skilled practitioner can readily determine the extracellular and transmembrane domains of the FGFR3 by aligning it with known RPTK (receptor protein tyrosine kinases) amino acid sequences for which these domains have been delineated. Alternatively, the hydrophobic transmembrane domain can be readily delineated based on a hydrophobicity plot of the polypeptide sequence. The extracellular domain is N-terminal to the transmembrane domain.

The term “epitope” is meant to refer to that portion of any molecule capable of being bound by an antibody or a fragment thereof which can also be recognized by that antibody. Epitopes or antigenic determinants usually consist of chemically active surface groupings of molecules such as amino acids or sugar side chains and have specific three-dimensional structural characteristics as well as specific charge characteristics.

An “antigen” is a molecule or a portion of a molecule capable of being bound by an antibody which is additionally capable of inducing an animal to produce antibody capable of binding to an epitope of that antigen. An antigen may have one or more than one epitope. The specific reaction referred to above is meant to indicate that the antigen will react, in a highly selective manner, with its corresponding antibody and not with the multitude of other antibodies which may be evoked by other antigens.

A “neutralizing antibody” as used herein refers to a molecule having an antigen-binding site to a specific receptor capable of reducing or inhibiting (blocking) activity or signaling through a receptor, as determined by in vivo or in vitro assays, as per the specification.

A “monoclonal antibody” or “mAb” is a substantially homogeneous population of antibodies to a specific antigen. mAbs may be obtained by methods known to those skilled in the art. See, for example Kohler et al (1975); U.S. Pat. No. 4,376,110; Ausubel et al (1987-1999); Harlow et a. (1988); and Colligan et al (1993), the contents of which references are incorporated entirely herein by reference. The mAbs of the present invention may be of any immunoglobulin class including IgG, IgM, IgE, IgA, and any subclass thereof. A hybridoma producing an mAb may be cultivated in vitro or in vivo. High titers of mAbs can be obtained in vivo production where cells from the individual hybridomas are injected intraperitoneally into pristine-primed Balb/c mice to produce ascites fluid containing high concentrations of the desired mabs. MAbs of isotype IgM or IgG may be purified from such ascites fluids, or from culture supernatants, using column chromatography methods well known to those of skill in the art.

Chimeric antibodies are molecules, the different portions of which are derived from different animal species, such as those having a variable region derived from a murine mAb and a human immunoglobulin constant region. Antibodies which have variable region framework residues substantially from human antibody (termed an acceptor antibody) and complementarity determining regions substantially from a mouse antibody (termed a donor antibody) are also referred to as humanized antibodies. Chimeric antibodies are primarily used to reduce immunogenicity in application and to increase yields in production, for example, where murine mAbs have higher yields from hybridomas but higher immunogenicity in humans, such that human/murine chimeric mAbs are used. Chimeric antibodies and methods for their production are known in the art (Better et al, 1988; Cabilly et al, 1984; Harlow et al, 1988; Liu et al, 1987; Morrison et al, 1984; Boulianne et al, 1984; Neuberger et al, 1985; Sahagan et al, 1986; Sun et al, 1987; Cabilly et al; European Patent Applications 125023, 171496, 173494, 184187, 173494, PCT patent applications WO 86/01533, WO 97/02671, WO 90/07861, WO 92/22653 and U.S. Pat. Nos. 5,693,762, 5,693,761, 5,585,089, 5,530,101 and 5,225,539). These references are hereby incorporated by reference.

Besides the conventional method of raising antibodies in vivo, antibodies can be generated in vitro using phage display technology. Such a production of recombinant antibodies is much faster compared to conventional antibody production and they can be generated against an enormous number of antigens. In contrast, in the conventional method, many antigens prove to be non-immunogenic or extremely toxic, and therefore cannot be used to generate antibodies in animals. Moreover, affinity maturation (i.e., increasing the affinity and specificity) of recombinant antibodies is very simple and relatively fast. Finally, large numbers of different antibodies against a specific antigen can be generated in one selection procedure. To generate recombinant monoclonal antibodies one can use various methods all based on phage display libraries to generate a large pool of antibodies with different antigen recognition sites. Such a library can be made in several ways: One can generate a synthetic repertoire by cloning synthetic CDR3 regions in a pool of heavy chain germline genes and thus generating a large antibody repertoire, from which recombinant antibody fragments with various specificities can be selected. One can use the lymphocyte pool of humans as starting material for the construction of an antibody library. It is possible to construct naive repertoires of human IgM antibodies and thus create a human library of large diversity. This method has been widely used successfully to select a large number of antibodies against different antigens. Protocols for bacteriophage library construction and selection of recombinant antibodies are provided in the well-known reference text Current Protocols in Immunology, Colligan et al (Eds.), John Wiley & Sons, Inc. (1992-2000), Chapter 17, Section 17.1.

In another aspect, the present invention provides methods of preventing, attenuating or treating the symptoms of a T-cell mediated inflammatory or autoimmune disease comprising administering a pharmaceutical composition comprising a therapeutically effective amount of a FGFR3 specific soluble receptor. A soluble receptor, also known as a secreted receptor, of the present invention comprises FGFR3 extracellular ligand binding sequences. The soluble receptor is able to freely circulate in the body and is useful for targeting, for example, a FGFR3 ligand. Without wishing to be bound by theory the soluble receptor binds the ligand, effectively inactivating it, since the FGFR3 ligand is then no longer able to bind with its biologic target in the body. An even more potent inhibitor consists of two soluble receptors fused together to a specific portion of an immunoglobulin molecule (Fc fragment). This produces a dimer, known as a fusion protein, composed of two soluble receptors which have a high affinity for the target, and a prolonged half-life. An example of this type of molecule is Enbrel® (etanercept) a dimeric fusion protein consisting of the extracellular ligand-binding portion of the human 75 kilodalton (p75) tumor necrosis factor receptor (TNFR) linked to the Fc portion of human IgGl, for the treatment of rheumatoid arthritis. The soluble receptor is produced by methods known in the art, including recombinant DNA technology and enzymatic techniques. In another aspect, the present invention provides a method of treating a T cell mediated inflammatory or autoimmune disease by administering a composition comprising at least one therapeutically effective FGFR3 inhibitor wherein the inhibitor is a FGFR3 specific small organic molecule tyrosine kinase inhibitor, and a pharmaceutically acceptable carrier. According to certain embodiments of the present invention an organic tyrosine kinase inhibitor (TKI) having FGFR3 specificity is useful for preventing, attenuating or treating a T cell mediated inflammatory or autoimmune disease. Non-limiting examples of organic tyrosine kinase inhibitors include small molecule organic tyrosine kinase inhibitors such as SU10991, SU5402 and PD173074 (Paterson, et al. 2004; Grand, et al, 2004)

In yet another embodiment, the present invention provides a method of treating a T cell mediated inflammatory or autoimmune disease by administering a pharmaceutical composition comprising at least one FGFR3 inhibitor wherein the inhibitor is a FGFR3 specific peptide inhibitor, and a pharmaceutically acceptable carrier. A peptide inhibitor includes FGFR3 specific peptides, peptide analogs having amino acid sequence derived from the extracellular portion of the fibroblast growth factor receptor 3 (FGFR3) and peptidomimetics based on the structure of such peptides. The peptidomimetic of the invention may be similar in structure to AHNP, the small molecule form of an anti-HER2/neu peptidomimetic that has activity similar to the full monoclonal antibody Herceptin (Zhang et al., 2000).

In yet another embodiment, the present invention provides a method of treating a T cell mediated inflammatory or autoimmune disease by administering a pharmaceutical composition comprising at least one FGFR3 inhibitor wherein the inhibitor is a FGFR3 specific RNA inhibitor, and a pharmaceutically acceptable carrier. RNA inhibition (RNAi) is based on antisense modulation of FGFR3 in cells and tissues comprising contacting the cells and tissues with at least one antisense compound, including but not limited to double stranded RNA, (dsRNA), small interfering RNA (siRNA), ribozymes and locked nucleic acids (LNAs). Antisense (AS) technology and its enormous therapeutic potential has been reviewed extensively (Milhavet, 2003; Oplanska, 2002). In certain specific embodiments the RNA inhibiting molecule is an antisense oligonucleotide or an oligonucleotide mimetic comprising from about 8 to about 50 nucleotides. International patent application WO 03/023004 teaches antisense compounds, compositions and methods for modulating the expression of FGFR3. Methods of using these compounds for diseases associated with FGFR3, such as skeletal disorders and certain cancers, are disclosed therein.

In yet another embodiment, the present invention provides a method of treating a T cell mediated inflammatory or autoimmune disease by administering a pharmaceutical composition comprising at least one FGFR3 inhibitor wherein the inhibitor is a FGFR3 specific antagonist ligand, and a pharmaceutically acceptable carrier. For example, copending application, PCT publication WO 03/094835, assigned to the assignee of the present invention, discloses FGF9 variant ligands having anatgonistic activity toward FGFR3.

In yet another embodiment the present invention provides a method of treating a T cell mediated inflammatory or autoimmune disease by administering a pharmaceutical composition comprising at least one FGFR3 inhibitor wherein the inhibitor is a FGFR3 specific inhibitor of heparan sulfate binding. In certain embodiments the heparan sulfate binding inhibitor is a natural or a synthetic compound including heparin and mimetics thereof.

In another embodiment, the present invention provides a method of treating a T cell mediated inflammatory or autoimmune disease by administering a pharmaceutical composition comprising at least one FGFR3 inhibitor wherein the inhibitor is a DNA vaccine encoding FGFR3 or a fragment thereof, and a pharmaceutically acceptable carrier. DNA vaccination provides an effective means of long term antigen expression in vivo for the generation of both humoral and cellular immune responses. According to various embodiments of the present invention the DNA vaccines encode active fragments of FGFR3, set forth in SEQ ID NO:75. The preferred polynucleotide fragments encode the extracellular domain polypeptide of FGFR3, in particular amino acids 1-370 or fragments thereof. The FGFR3 according to the present invention is preferably human, however other mammalian FGFR3 proteins are within the scope of the invention.

The amino acid sequence of FGFR3 IIIb isoform (NCBI access no: P22607) is denoted herein SEQ ID NO:75.

MGAPACALAL CVAVAIVAGA SSESLGTEQR VVGRAAEVPG
PEPGQQEQLV FGSGDAVELS CPPPGGGPMG PTVWVKDGTG
LVPSERVLVG PQRLQVLNAS HEDSGAYSCR QRLTQRVLCH
FSVRVTDAPS SGDDEDGEDE AEDTGVDTGA PYWTRPERMD
KKLLAVPAAN TVRFRCPAAG NPTPSISWLK NGREFRGEHR
IGGIKLRHQQ WSLVMESVVP SDRGNYTCVV ENKFGSIRQT
YTLDVLERSP HRPILQAGLP ANQTAVLGSD VEFHCKVYSD
AQPHIQWLKH VEVNGSKVGP DGTPYVTVLK VSLESNASMS
SNTPLVRIAR LSSGEGPTLA NVSELELPAD PKWELSRARL
TLGKPLGEGC FGQVVMAEAI GIDKDRAAKP VTVAVKMLKD
DATDKDLSDL VSEMEMMKMI GKHKNIINLL GACTQGGPLY
VLVEYAAKGN LREFLRARRP PGLDYSFDTC KPPEEQLTFK
DLVSCAYQVA RGMEYLASQK CIHRDLAARN VLVTEDNVMK
IADFGLARDV HNLDYYKKTT NGRLPVKWMA PEALFDRVYT
HQSDVWSFGV LLWEIFTLGG SPYPGIPVEE LFKLLKEGHR
MDKPANCTHD LYMIMRECWH AAPSQRPTFK QLVEDLDRVL
TVTSTDEYLD LSAPFEQYSP GGQDTPSSSS SGDDSVFAHD
LLPPAPPSSG GSRT

The corresponding polynucleotide sequence (NCBI access no: AF245114) is denoted herein SEQ ID NO:76.

In another aspect the present invention provides a method of treating a T cell mediated inflammatory or autoimmune disease by administering a pharmaceutical composition comprising at least one FGFR3 inhibitor wherein the inhibitor is a FGFR3 specific heparin inhibitor or a heparin mimetic inhibitor. Heparin and other proteoglycans are known to be essential to growth factor binding to a receptor. In a non-limiting example, the present invention provides a heparin-like, polyanionic compound able to compete with heparin for binding to FGFR3.

### Pharmacology

The present invention also contemplates pharmaceutical formulations, both for veterinary and for human medical use, which comprise as the active agent one or more of the FGFR3 inhibitors described in the invention, for the manufacture of a medicament for the treatment or prophylaxis of the conditions variously described herein.

In such pharmaceutical and medicament formulations, the active agent preferably is utilized together with one or more pharmaceutically acceptable carrier(s) therefore and optionally any other therapeutic ingredients. The carrier(s) must be pharmaceutically acceptable in the sense of being compatible with the other ingredients of the formulation and not unduly deleterious to the recipient thereof. The active agent is provided in an amount effective to achieve the desired pharmacological effect, as described above, and in a quantity appropriate to achieve the desired daily dose.

Typically, the molecules of the present invention comprising the antigen binding portion of an antibody or comprising another polypeptide including a peptidomimetic, antagonistic ligand or soluble receptor or an organic molecule or polynucleotide will be suspended in a sterile saline solution for therapeutic uses. The pharmaceutical compositions may alternatively be formulated to control release of active ingredient (molecule comprising the antigen binding portion of an antibody) or to prolong its presence in a patient's system. Numerous suitable drug delivery systems are known and include, e.g., implantable drug release systems, hydrogels, hydroxymethylcellulose, microcapsules, liposomes, microemulsions, microspheres, and the like. Controlled release preparations can be prepared through the use of polymers to complex or adsorb the molecule according to the present invention. For example, biocompatible polymers include matrices of poly(ethylene-co-vinyl acetate) and matrices of a polyanhydride copolymer of a stearic acid dimer and sebaric acid (Sherwood et al, 1992). The rate of release of the molecule according to the present invention, i.e., of an antibody or antibody fragment, from such a matrix depends upon the molecular weight of the molecule, the amount of the molecule within the matrix, and the size of dispersed particles (Saltzman et al., 1989 and Sherwood et al., 1992). Other solid dosage forms are described in Ansel et al., 1990 and Gennaro, 1990.

The pharmaceutical composition of this invention may be administered by any suitable means, such as orally, topically, intranasally, subcutaneously, intramuscularly, intravenously, intra-arterially, intraarticulary, intralesionally or parenterally. Ordinarily, intravenous (i.v.), intraarticular, topical or parenteral administration will be preferred.

It will be apparent to those of ordinary skill in the art that the therapeutically effective amount of the molecule according to the present invention will depend, inter alia upon the administration schedule, the unit dose of molecule administered, whether the molecule is administered in combination with other therapeutic agents, the immune status and health of the patient, the therapeutic activity of the molecule administered and the judgment of the treating physician. As used herein, a “therapeutically effective amount” refers to the amount of a molecule required to alleviate one or more symptoms associated with a disorder being treated over a period of time.

Although an appropriate dosage of a molecule of the invention varies depending on the administration route, type of molecule (polypeptide, polynucleotide, organic molecule etc.) age, body weight, sex, or conditions of the patient, and should be determined by the physician in the end, in the case of oral administration, the daily dosage can generally be between about 0.01 mg to about 500 mg, preferably about 0.01 mg to about 50 mg, more preferably about 0.1 mg to about 10 mg, per kg body weight. In the case of parenteral administration, the daily dosage can generally be between about 0.001 mg to about 100 mg, preferably about 0.001 mg to about 10 mg, more preferably about 0.01 mg to about 1 mg, per kg body weight. The daily dosage can be administered, for example in regimens typical of 1-4 individual administration daily. Other preferred methods of administration include intraarticular administration of about 0.01 mg to about 100 mg per kg body weight. Various considerations in arriving at an effective amount are described, e.g., in Goodman and Gilman's: The Pharmacological Bases of Therapeutics, 8th ed., Pergamon Press, 1990; and Remington's Pharmaceutical Sciences, 17th ed., Mack Publishing Co., Easton, Pa., 1990.

The molecules of the present invention as active ingredients are dissolved, dispersed or admixed in an excipient that is pharmaceutically acceptable and compatible with the active ingredient as is well known. Suitable excipients are, for example, water, saline, phosphate buffered saline (PBS), dextrose, glycerol, ethanol, or the like and combinations thereof. Other suitable carriers are well known to those in the art. (See, for example, Ansel et al., 1990 and Gennaro, 1990). In addition, if desired, the composition can contain minor amounts of auxiliary substances such as wetting or emulsifying agents, pH buffering agents.

The combined treatment of one or more of the molecules of the invention with an anti-inflammatory drug such as methotrexate or glucocorticoids may provide a more efficient treatment for inhibiting FGFR3 activity. In one embodiment, the pharmaceutical composition comprises the antibody, an anti-inflammatory drug and a pharmaceutically acceptable carrier.

### Polynucleotides

The term “nucleic acid” and “polynucleotides” refers to molecules such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

Within the scope of the present invention is a nucleic acid molecule encoding a FGFR3 inhibitor useful for the preparation of a medicament for the treatment for the conditions described herein. The nucleic acid molecule contains a nucleotide sequence having at least 75% sequence identity, preferably about 90%, and more preferably about 95% identity to the above encoding nucleotide sequence set forth in any one of SEQ ID NOS: 57-74, as would well understood by those of skill in the art. In the hypervariable regions of the heavy chain and light chain, the nucleic acid molecule contains a nucleotide sequence having at least 50% sequence identity, preferably about 70% and more preferably about 80% identity to the molecules set forth in any one of SEQ ID NOs: 39-56.

The invention also provides nucleic acids that hybridize under high stringency conditions to polynucleotides set forth in any one of SEQ ID NOs: 57-74 or the complement thereof. As used herein, highly stringent conditions are those which are tolerant of up to about 5%-25% sequence divergence, preferably about 5%-15%. Without limitation, examples of highly stringent (−10° C. below the calculated Tm of the hybrid) conditions use a wash solution of 0.1×SSC (standard saline citrate) and 0.5% SDS at the appropriate Ti below the calculated Tm of the hybrid. The ultimate stringency of the conditions is primarily due to the washing conditions, particularly if the hybridization conditions used are those which allow less stable hybrids to form along with stable hybrids. The wash conditions at higher stringency then remove the less stable hybrids. A common hybridization condition that can be used with the highly stringent to moderately stringent wash conditions described above is hybridization in a solution of 6×SSC (or 6×SSPE), 5× Denhardt's reagent, 0.5% SDS, 100 μg/ml denatured, fragmented salmon sperm DNA at an appropriate incubation temperature Ti. See generally Sambrook et al., Molecular Cloning: A Laboratory Manual, 2d edition, Cold Spring Harbor Press (1989)) for suitable high stringency conditions.

Stringency conditions are a function of the temperature used in the hybridization experiment and washes, the molarity of the monovalent cations in the hybridization solution and in the wash solution(s) and the percentage of formamide in the hybridization solution. In general, sensitivity by hybridization with a probe is affected by the amount and specific activity of the probe, the amount of the target nucleic acid, the detectability of the label, the rate of hybridization, and the duration of the hybridization. The hybridization rate is maximized at a Ti (incubation temperature) of 20-25° C. below Tm for DNA:DNA hybrids and 10-15° C. below Tm for DNA:RNA hybrids. It is also maximized by an ionic strength of about 1.5M Na⁺. The rate is directly proportional to duplex length and inversely proportional to the degree of mismatching.

Specificity in hybridization, however, is a function of the difference in stability between the desired hybrid and “background” hybrids. Hybrid stability is a function of duplex length, base composition, ionic strength, mismatching, and destabilizing agents (if any).

The Tm of a perfect hybrid may be estimated for DNA:DNA hybrids using the equation of Meinkoth et al (1984), as

Tm=81.5° C.+16.6 (log M)+0.41 (%GC)−0.61 (% form)−500/L

and for DNA:RNA hybrids, as

Tm=79.8° C.+18.5 (log M)+0.58 (%GC)−11.8 (%GC)²−0.56 (% form)−820/L

where M, molarity of monovalent cations, 0.01-0.4 M NaCl,

%GC, percentage of G and C nucleotides in DNA, 30%-75%,

% form, percentage formamide in hybridization solution, and

L, length hybrid in base pairs.

Tm is reduced by 0.5-1.5° C. (an average of 1° C. can be used for ease of calculation) for each 1% mismatching. The Tm may also be determined experimentally. As increasing length of the hybrid (L) in the above equations increases the Tm and enhances stability, the full-length rat gene sequence can be used as the probe.

Filter hybridization is typically carried out at 68° C., and at high ionic strength (e.g., 5-6×SSC), which is non-stringent, and followed by one or more washes of increasing stringency, the last one being of the ultimately desired high stringency. The equations for Tm can be used to estimate the appropriate Ti for the final wash, or the Tm of the perfect duplex can be determined experimentally and Ti then adjusted accordingly.

The invention also provides for conservative amino acid variants of the molecules. Variants according to the invention also may be made that conserve the overall molecular structure of the encoded proteins. Given the properties of the individual amino acids comprising the disclosed protein products, some rational substitutions will be recognized by the skilled worker. Amino acid substitutions, i.e. “conservative substitutions,” may be made, for instance, on the basis of similarity in polarity, charge, solubility, hydrophobicity, hydrophilicity, and/or the amphipathic nature of the residues involved.

In another embodiment of the present invention the FGFR3 inhibitor is a RNA molecule, for example a RNA molecule disclosed in PCT publication WO 03/023004.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### EXAMPLES

An important approach to inhibiting FGFR3 activity is the generation of reagents that block receptor signaling. Without wishing to be bound by theory, molecules which bind the extracellular domain of the receptor may inhibit the receptor by competing with FGF or heparin binding or, alternatively, by preventing receptor dimerization. Additionally, binding to the extracellular domain may accelerate receptor internalization and turnover. Molecules which block receptor activity intracellularly may do so by a number of mechanisms, including, without limitation, blocking the ATP binding site or by preventing phosphorylation.

All the experiments were performed under the supervision and guidelines of the Animal Welfare Committee.

### Example 1

### Collagen Induced Arthritis (CIA)

Murine collagen-induced arthritis (CIA) is considered a useful model for studying human RA since the two diseases share numerous pathologic, immunological, and genetic features. The CIA model in mice results in a symmetric polyarthritis in which bone and cartilage erosion typically occur, 2-4 weeks after immunization with naive type II collagen on complete Freud's adjuvant (CFA).

Male DBA/l mice (8-10 weeks old) were subject to an intradermal injection at the base of the tail with 200 μg type II collagen purified from bovine articular cartilage emulsified in CFA. The mice received a booster injection of 200 μg type II collagen emulsified in CFA three weeks after the first dose.

The mice were checked daily and each animal with edema in one or more limbs was randomly assigned to one of several groups for further treatment. The thickness of each affected hind paw was measured with microcalipers. The results are expressed as a direct measure of paw width in millimeters.

### Example 2

### Treatment of CIA Mice with FGFR3 Inhibitors

Each mouse was injected intraperitoneally on the day following disease onset (day 1) with 100 ug anti-FGFR3-ScFv or anti-FGFR3 Fab′ or 20 mg/kg of a FGFR3 specific tyrosine kinase (TK) inhibitor (SU5402, Calbiochem, La Jolla, Calif.), followed by daily injections with 300 μg anti-FGFR3 ScFv or anti-FGFR3 Fab′ or with 20 mg/kg SU5402.

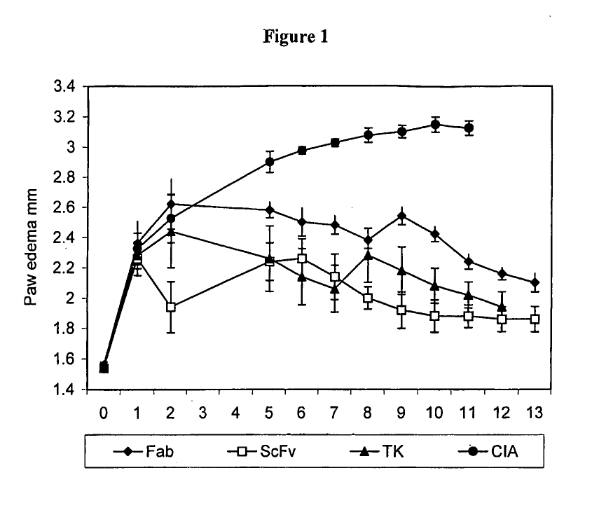

FIG. 1 shows the results of the inflammatory response to the various FGFR3 inhibitors. Day 0 refers to the day of boost. The untreated animals () show a steady increase in paw edema until day 5 where it begins to stabilize at approximately 3 mm. All the treated animal responded to the anti-FGFR3 treatment. The anti-FGFR3 scFv treated animals (▭) showed the greatest reduction in paw edema over a 13 day period, to approximately 1.9 mm. The anti-FGFR3 Fab treated animals (♦) showed a significant reduction as did the SU5402 (TK) inhibitor treated animals (▴).

### Example 3

### Delayed Type Hypersensitivity (DTH) Assessments

The mouse model for cutaneous delayed type hypersensitivity reactions was used to investigate the effects of FGFR3 inhibitors on induced skin inflammation. Oxazolone solutions (2% and 0.5%) were prepared by dissolving 200 and 50 mg, respectively, of oxazolone in 8 ml of acetone and 2 ml of olive oil. Mice were challenged with oxazolone by topical application onto the abdomen of each mouse (100 μl of 2% oxazolone) followed by 10 μl of 0.5% oxazolone on the right ear after 6 days. Differences between right and left ear thickness, indicating DTH development, were measured after 24 hours using a microcaliper.

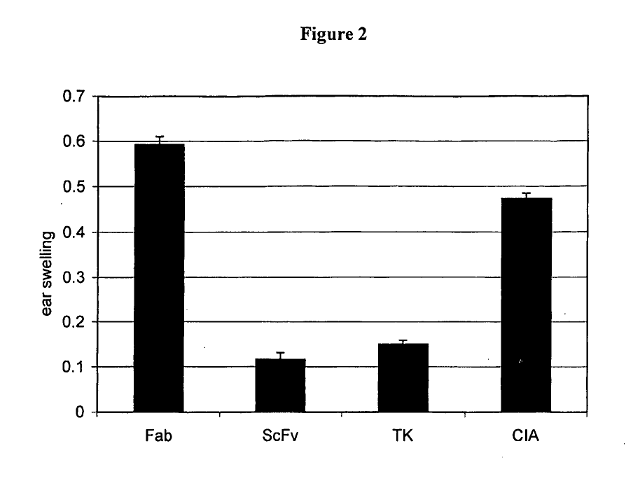

FIG. 2 shows the results of the DTH assay. The CIA mice showed a strong inflammatory reaction to the collagen. The scFv and SU5402 (TK) treated mice exhibited no induction of edema. The anti-FGFR3 Fab treated mice exhibit a strong reaction which, without wishing to be bound to a certain theory, may indicate an immune reaction to the Fab itself. The antibody is a fully human protein that may elicit a reaction in this particular assay. These studies show that systemic administration of an anti-FGFR3 ScFv or TK inhibitor do not induce skin inflammation in this model of experimentally-induced skin inflammation in mice.

### Example 4

### Interferon-γ (INF-γ) Secretion Assay

Interferon-γ secretion was determined in an ELISA assay. Leukocytes were isolated from mice spleens. One million (10⁶) cells were incubated in medium containing serum for 24 hour, in a 24 well plate coated with anti-CD3 and anti-CD28 antibodies. Supernatant from each sample was collected and assayed for presence of INF-γ. The INF-γ, assay was performed as follows: 96 wells plates were coated with a monoclonal anti-INF-τ dissolved in boric buffer. Different volumes of supernatant were added to each well and incubated for 1 hour. IFN-γ was detected using anti-INF-γ mAb-biotin followed with alkaline phosphatase conjugated Strepavidin. PNPP substrate was added and the color intensity measured in ELISA reader at Absorbance 405 nm. The anti-FGFR3 Fab and scFv antibodies showed reduced IFN-γ secretion.

### Example 5

### DNA Vaccines

PCT patent publications WO 00/06203 and WO 01/57056 describe a method for inducing protective immunity against multiple sclerosis and rheumatoid arthritis, respectively, the method comprising intramuscularly administering to a subject a naked DNA nucleic acid construct encoding a cytokine. In this experiment a naked DNA nucleic acid construct encoding FGFR3 extracellular domain or an active fragment thereof is administered in an animal model mimicking multiple sclerosis, as described in example 6 or in a experimental rat model of rheumatoid arthritis (Lider et al., 1987) or other animal model of T cell mediated autoimmune diseases. Rats are immunized subcutaneously in the base-tail with 0.1 ml of CFA supplemented with 10 mg/ml heat-killed Mycobacterium tuberculosis H37Ra in oil (Difco Laboratories Inc., Detroit, Mich.). Rats are monitored for clinical signs daily by an observer blind to the treatment protocol.

### Example 6

### Experimental Autoimmune Encephalomyelitis (EAE)

Experimental allergic encephalomyelitis (EAE) is an autoimmune neurological disease elicited by sensitization of the animals to myelin basic protein from the central nervous system. EAE is considered by many to represent a model of the human disease multiple sclerosis. The ability of compounds of the FGFR3 inhibitors to prevent or attenuate the clinical symptoms of this autoimmune disease is tested.

EAE is induced in Lewis rats in which the disease displays onset of symptoms around day 10 after induction and spontaneous recovery around 18 days after induction of the disease. Eight-week old female Lewis rats are used in this model. The animals (5 per cage) are maintained on a 12-hour light/12 hour dark regimen, with food and water ad libitum. EAE was induced in these animals by immunization with purified guinea pig myelin basic protein emulsified in complete Freund's adjuvant. Guinea pig myelin basic protein (MBP) is prepared from spinal cord homogenates defatted with chloroform/ethanol and the isolated protein was purified using ion exchange chromatography. Each animal receives 50 micrograms of the purified protein. A solution of MBP (0.5 mg/ml) is emulsified with an equal volume of Complete Freund's Adjuvant containing 4 mg/ml of mycobacterium tuberculosis, and each animal receives 100 microliters (50 ul in each hind foot pad). Animals are treated with a single injection of anti-FGFR3 antibody or vehicle control administered intravenously in a volume of 2 ml. The time of treatment is varied from day 10 to day 18, post induction of disease, with five animals per group.

The present invention is exemplified by certain animal disease models. These models are intended as a non-limitative example used for illustrative purposes of the principles of the present invention. Other animal models, such as those described in Gregersen et al (2004), are useful for demonstrating the principles described herein.

### REFERENCES

Ansel et al, Pharmaceutical Dosage Forms and Drug Delivery Systems, 5th Ed. (Lea & Febiger 1990)
Ausubel, et al (Eds), Current Protocols in Molecular Biology, John Wiley & Sons, Inc. (New York) (1987-1999)
Better et al, ″, Science 240d(4855):1041-1043 (1988)
Boulianne, et al, Nature 312(5995):643-646 (1984)
Colligan, et al (eds.), Current Protocols in Immunology, John Wiley & Sons, Inc. (New York) (1992-2000)
Gennaro (ed.), Remington's Pharmaceutical Sciences, 18^{th} Ed. (Mack Publishing Co., 1990)
Grand E K, et al., Leukemia 18(5):962-66 (2004)
Gregerson, J W, et al., Tiss. Antigens 63:383-394 (2004)
Harlow, et al, Antibodies: A Laboratory Manual, CSHL (Cold Spring Harbor, N.Y.) (1988)
Kohler and Milstein, Nature 256(5517):495-497 (1975)
Liu et al, PNAS USA, 84(10):3439-3443 (1987)
Manabe, N, et al., Rheumatology 38:714-20, (1999)
Milhavet, O, et al., Pharmacol. Rev. 55(4):629-48 (2003)
Morrison et al., PNAS USA 81(21):6851-6855 (1984)
Muller, et al, FEBS Letters, 432:45-49 (1998)
Nepom, G T, Curr Opin Immun 14:812-815 (2002)
Neuberger et al, Nature 314(6008):268-270 (1985)
Opalinska J B and Gewirtz A M, Nat Rev Drug Discov. 1(7):503-14 (2002)
Ornitz, Novartis Found Symp 232:63-76; discussion 76-80, 272-82 (2001)
Paleolog, E M, Arthritis Res., 4(suppl 3):S81-90 (2002)
Paterson, J L, et al., Br. J. Haematol. 124(5):595-603 (2004)
Saltzman et al, Biophys. J. 55:163 (1989)
Sambrook, J, Fritsch, E. F. and Maniatis, T. (1989) Molecular Cloning: A laboratory manual, Cold Spring Harbor Laboratory Press, Cold Spring Harbor, USA.
Simon, J A, Rev Invest Clin 53:452-459, (2001)
Sherwood, et al., Biotechnology, 10(11):1446-9 (1992)
Walsh, D A and Haywood, L, Curr Opin Investig Drugs, 2:1054-63 (2001)
Zhang, H T, et al., Drug News Perspect 13(6):325-9 (2000)

1. A method for preventing or treating a T cell mediated inflammatory or autoimmune disease comprising administering to an individual in need thereof a therapeutically effective amount of at least one FGFR 3 inhibitor and a pharmaceutically acceptable carrier.

2. The method according to claim 1 wherein said at least one FGFR3 inhibitor is selected from a group consisting of a molecule comprising the antigen-binding portion of an antibody which has a specific affinity for FGFR3, a FGFR3 specific small organic molecule tyrosine kinase inhibitor, a FGFR3 specific soluble receptor, a FGFR3 specific peptide or peptidomimetic, a FGFR3 specific RNA inhibitor, a FGFR3 specific antagonist ligand and a DNA vaccine encoding FGFR3 or a fragment thereof, an FGFR3 specific inhibitor of heparan sulfate binding.

3. The method according to claim 2 wherein said at least one FGFR3 inhibitor is a molecule comprising the antigen-binding portion of an antibody which has a specific affinity for the extracellular domain of FGFR3.

4. The method according to claim 3 wherein said molecule comprising the antigen-binding portion of an antibody which has a specific affinity for FGFR3 is a monoclonal antibody or a proteolytic fragment thereof.

5. The method according to claim 4 wherein said monoclonal antibody or proteolytic fragment thereof is an anti-FGFR3 Fab.

6. The method according to claim 34 wherein said molecule comprising the antigen-binding portion of an antibody which has a specific affinity for FGFR3 is a single chain Fv set forth in SEQ ID NO:37.

7. The method according to claim 3 wherein said molecule comprising the antigen-binding portion of an antibody which has a specific affinity for FGFR3 comprising a V_{H}-CDR3 region selected from a group consisting of polypeptides set forth in anyone of SEQ ID NOS:1-9 and a V_{L}-CDR3 region selected from a group consisting of polypeptides set forth in anyone of SEQ ID NOS:10-18.

8. The method according to claim 7 wherein said molecule comprising the antigen-binding portion of an antibody which has a specific affinity for FGFR3 comprising a V_{H}-CDR3 region set forth in SEQ ID NO:1 and a V_{L}-CDR3 region set forth in SEQ ID NO:10.

9. The method according to claim 3 wherein said molecule comprising the antigen-binding portion of an antibody which has a specific affinity for FGFR3 comprising a V_{H} region selected from a group of polypeptides set forth in anyone of SEQ ID NOS:19-27 and a V_{L} region selected from the group of polypeptides set forth in anyone of SEQ ID NOS:28-36.

10. The method according to claim 9 wherein said molecule comprising the antigen-binding portion of an antibody which has a specific affinity for FGFR3 comprising a V_{H} region set forth in SEQ ID NO:19 and a V_{L} region set forth in SEQ ID NO:28.

11. The method according to claim 2 wherein said at least one FGFR3 inhibitor is a FGFR3 specific small organic molecule tyrosine kinase inhibitor.

12. The method according to claim 1 wherein the T cell mediated inflammatory autoimmune disease is selected from rheumatoid arthritis, collagen II arthritis, multiple sclerosis, systemic lupus erythematosus, psoriasis, juvenile onset diabetes, Sjogren's disease, thyroid disease, sarcoidosis, autoimmune uveitis, inflammatory bowel disease (Crohn's and ulcerative colitis), celiac disease and myasthenia gravis.

13. The method according to claim 12 wherein the T cell mediated inflammatory autoimmune disease is rheumatoid arthritis.

14. Use of at least one FGFR 3 inhibitor for the preparation of a medicament for preventing and treating a T cell mediated inflammatory autoimmune disease.

15. Use according to claim 14 wherein said at least one FGFR3 inhibitor is selected from a group consisting of a molecule comprising the antigen-binding portion of an antibody which has a specific affinity for FGFR3, a FGFR3 specific small organic molecule tyrosine kinase inhibitor, a FGFR3 specific soluble receptor, a FGFR3 specific peptide or peptidomimetic, a FGFR3 specific RNA inhibitor, a FGFR3 specific antagonist ligand and a DNA vaccine encoding FGFR3 or a fragment thereof.

16. Use according to claim 15 wherein said at least one FGFR3 inhibitor is a molecule comprising the antigen-binding portion of an antibody which has a specific affinity for the extracellular domain of FGFR3.

17. Use according to claim 15 wherein said molecule comprising the antigen-binding portion of an antibody which has a specific affinity for FGFR3 is a monoclonal antibody or proteolytic fragment thereof.

18. Use according to claim 17 wherein said monoclonal antibody or proteolytic fragment thereof is an anti-FGFR3 Fab.

19. Use according to claim 15 wherein said molecule comprising the antigen-binding portion of an antibody which has a specific affinity for FGFR3 is a single chain Fv set forth in SEQ ID NO:37.

20. Use according to claim 15 wherein said molecule comprising the antigen-binding portion of an antibody which has a specific affinity for FGFR3 comprising a V_{H}-CDR3 region selected from a group consisting of polypeptides set forth in anyone of SEQ ID NOS:1-9 and a V_{L}-CDR3 regions selected from a group consisting of polypeptides set forth in anyone of SEQ ID NOS:10-18.

21. Use according to claim 20 wherein said molecule comprising the antigen-binding portion of an antibody which has a specific affinity for FGFR3 comprising a V_{H}-CDR3 region set forth in SEQ ID NO:1 and a V_{L}-CDR3 region set forth in SEQ ID NO:10.

22. Use according to claim 15 wherein said molecule comprising the antigen-binding portion of an antibody which has a specific affinity for FGFR3 comprising a V_{H} region selected from a group of polypeptides set forth in anyone of SEQ ID NOS:19-27 and a V_{L} region selected from the group of polypeptides set forth in anyone of SEQ ID NOS:28-36.

23. Use according to claim 22 wherein said molecule comprising the antigen-binding portion of an antibody which has a specific affinity for FGFR3 comprising a V_{H} region set forth in SEQ ID NO:19 and a V_{L} region set forth in SEQ ID NO:28.

24. Use according to claim 15 wherein said at least one FGFR3 inhibitor is a FGFR3 specific small organic molecule tyrosine kinase inhibitor.

25. Use according to claim 14 wherein the T cell mediated inflammatory autoimmune disease is selected from rheumatoid arthritis, collagen II arthritis, multiple sclerosis, systemic lupus erythematosus, psoriasis, juvenile onset diabetes, Sjogren's disease, thyroid disease, sarcoidosis, autoimmune uveitis, inflammatory bowel disease (Crohn's and ulcerative colitis), celiac disease and myasthenia gravis.

26. Use according to claim 25 wherein the T cell mediated inflammatory autoimmune disease is rheumatoid arthritis.
